(19) United States
(12) Patent Application Publication
(10) Pub. No.: US 2026/0233006 A1
(43) Pub. Date: Aug. 13, 2026

(54) Methods to Remove Electrical Stimulation Artifacts in Neural Signals

(71) Applicant: Washington University, St. Louis, MO (US)

(72) Inventors: Peter Brunner, St. Louis, MO (US); Tao Xie, St. Louis, MO (US); Phillip Demarest, St. Louis, MO (US); Jon Willie, St. Louis, MO (US); Eric Leuthardt, St. Louis, MO (US)

(73) Assignee: Washington University, St. Louis, MO (US)

(21) Appl. No.: 19/537,450

(22) Filed: Feb. 11, 2026

Related U.S. Application Data
(60) Provisional application No. 63/757,070, filed on Feb. 11, 2025.

(51) Int. Cl. A61N 1/36

(52) U.S. Cl. CPC A61N 1/36082; A61N 1/36025

(57) ABSTRACT

The present disclosure provides methods to remove stimulation artifacts from neurophysiological activity data during electrical brain stimulation. The disclosed method includes a baseline-integrated approach, Linear Baseline-Integrated Removal of Artifacts, that preserves broadband high-frequency activity during electrical stimulation.

### CROSS-REFERENCE TO RELATED APPLICATIONS

This application claims the benefit of priority to U.S. Provisional Application Ser. No. 63/757,070 filed on Feb. 11, 2025, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE DISCLOSURE

The present disclosure generally relates to electrical brain stimulation and methods for removing brain stimulation artifacts in neural signals.

### BACKGROUND

Electrical stimulation artifacts not only obscure physiological responses but also mimic neural activity through spectral leakage or temporal spread. This challenge is further exacerbated by the uncertainty principle, which temporally spreads residual artifacts when signals are filtered into the BHA range. Various methods have been proposed to eliminate stimulation artifacts; however, their efficacy remains limited due to the fundamental methodology issues. Existing artifact removal approaches fall into three main categories: interpolation, template subtraction, and model decomposition (FIG. 6A). Interpolation methods replace artifact-affected signals with modeled signals, such as linear interpolation, curve fitting, autoregressive modeling, or linear merging of surrounding signals. However, these techniques often introduce additional artifacts during the ‘interpolate’ period, distorting the underlying signal. Template subtraction methods rely on predefined artifact templates generated through trial averaging, manifold learning, or biophysical modeling. However, these methods struggle with variability in artifact shape across trials. For instance, neural recordings are typically sampled at rates too low (e.g., 1 kHz) to accurately capture the shape of stimulation artifacts resulting from 0.1-1 ms stimulation pulses. This variability limits the effectiveness of template subtraction methods. Model decomposition methods separate neural signals into distinct components using techniques such as independent component analysis (ICA), principal component analysis (PCA), dictionary learning, empirical mode decomposition, and matching-pursuit-based algorithm. Despite their promise, a lack of comprehensive understanding of individual neural signal components limits the applicability and reliability of these methods in practical settings.

Electrical brain stimulation is a powerful tool for modulating neural activity, widely utilized in neuroscience and neuroengineering research, as well as in clinical treatments for neurological and psychiatric disorders. Studying the neurophysiological effects of stimulation provides critical insights into brain function and is promising for the optimization of therapeutic interventions. Among these, broadband high-frequency activity (BHA, 70-170 Hz) is of particular interest as it reflects population-level neural activity closely linked to local multi-unit spiking. However, electrical stimulation artifacts-especially those overlapping with the BHA range-severely contaminate BHA signals, making it difficult to distinguish true neurophysiological responses from artifact-induced spectral distortions. As a result, most studies have been limited to analyzing post-stimulation BHA, leaving real-time BHA dynamics during stimulation largely unexplored. As shown conceptually in FIG. 1A, linear interpolation-a common artifact removal strategy-can introduce artificial broadband activity, distort true BHA responses and compromise the accuracy of neurophysiological measurements. This limitation leaves the real-time BHA dynamics occurring during electrical stimulation largely unknown, posing a major barrier to understanding how brain stimulation modulates ongoing neural activity at the population level.

Various methods have been proposed to eliminate stimulation artifacts. Existing artifact removal approaches mainly fall into three main categories: interpolation, template subtraction, and model decomposition (FIG. 1D).

Interpolation methods replace artifact-affected signals with modeled signals, such as linear interpolation, curve fitting, autoregressive modeling, or physiological interpolation by linear merging of surrounding signals. However, these techniques often introduce additional artifacts during the ‘interpolate’ period, which can distort the underlying neurophysiological signal.

Template subtraction methods rely on predefined artifact templates generated through trial averaging, manifold learning, or biophysical modeling. However, these methods struggle with variability in artifact shape across trials. For instance, neural recordings are typically sampled at rates too low (e.g., 1 kHz) to accurately capture the shape of stimulation artifacts resulting from 0.1-1 ms stimulation pulses. This variability limits the effectiveness of template subtraction methods.

Model decomposition methods separate neural signals into distinct components using techniques such as independent component analysis (ICA), principal component analysis (PCA), dictionary learning, empirical mode decomposition, and matching-pursuit-based algorithm. Despite their promise, a lack of comprehensive understanding of individual neural signal components limits the applicability and reliability of these methods in practical settings.

Early efforts to extract BHA responses during electrical stimulation have shown promise but remain limited by methodological challenges. For instance, one study employed physiological interpolation to extract BHA responses during 10 Hz stimulation, while other recent work applied a matching-pursuit-based algorithm to isolate early BHA responses during single-pulse electrical stimulation. However, reliably extracting BHA activity during electrical stimulation remains a significant challenge, particularly when stimulation frequencies overlap with the BHA range. While traditional artifact removal methods can reduce high-amplitude artifacts, residual components often persist. These residuals lead to spectral leakage and temporal smearing when signals are bandpass filtered, distorting true BHA and mimicking neurophysiological activity. These limitations underscore the crucial need for a robust methodology that can effectively isolate BHA signals during electrical stimulation, particularly during high-frequency stimulation.

### SUMMARY

Among the various aspects of the present disclosure is the provision of methods for removing brain stimulation artifacts in neural signals.

Included in the disclosure is a method to obtain neurophysiological data during an electrical stimulation, wherein the electrical stimulation introduces a stimulation artifact, the method comprising removal of the stimulation artifact with a Linear Baseline-Integrated Removal of Artifacts (LIBRA) approach. In one aspect, the LIBRA approach comprises: subtraction of a trial-averaged signal from an individual stimulation trial; an interpolation of a baseline period and an interpolation of a stimulation period with the stimulation artifact; extraction of a non-phased-locked response; subtractions of a baseline spectral response in the baseline period from a stimulation spectral response in the stimulation period, and; extraction of a neurophysiological signal, wherein subtracting the baseline spectral response eliminates the stimulation artifact from the stimulation period. In another aspect, the interpolation of the baseline period and the interpolation of the stimulation period are independently selected from methods comprising: a linear interpolation, a Crowther method, a polynomial curve fitting method, a matching pursuit method, and a wavelet interpolation method. In another aspect, the electrical brain stimulation is at a stimulation frequency comprising theta (4-7 Hz), alpha (8-12 Hz), low beta (13-20 Hz), high beta (20-30 Hz), low gamma (30-50 Hz), and broadband high-frequency activity (BHA; 70-170 Hz).

In one aspect, computer-implemented method to remove stimulation artifacts from a series of signal sets indicative of electrophysiological activity obtained during at least one stimulation trial. The method includes subtracting, using a computing device, a trial-averaged signal from each individual stimulation trial of the at least one stimulation trial; interpolating, using the computing device, the series of signals within a baseline period and interpolating within a stimulation period containing the stimulation artifacts for each individual stimulation trial of the at least one stimulation trial; extracting, using the computing device, a non-phased-locked response from the series of signals for each individual stimulation trial of the at least one stimulation trial; subtracting, using the computing device, a baseline spectral response within the baseline period from a stimulation spectral response within the stimulation period to produce a neurophysiological signal set for each individual stimulation trial of the at least one stimulation trial; and displaying, using the computing device, the neurophysiological signal set for each individual stimulation trial of the at least one stimulation trial, wherein each neurophysiological signal set corresponds to each signal set of the series of signal sets with stimulation artifacts removed. In some aspects, the method of interpolation of the baseline period and the method of interpolation of the stimulation period are independently selected from a linear interpolation method, a Crowther method, a polynomial curve fitting method, a matching pursuit method, and a wavelet interpolation method. In some aspects, the electrical brain stimulation is performed at a stimulation frequency comprising theta (4-7 Hz), alpha (8-12 Hz), low beta (13-20 Hz), high beta (20-30 Hz), low gamma (30-50 Hz), broadband high-frequency activity (BHA; 70-170 Hz), and any combination thereof. In some aspects,

In another aspect, a computer-implemented method of monitoring an efficacy of a neuromodulation therapy by transforming a series of signal sets indicative of electrophysiological activity obtained during at least one stimulation trial associated with the neuromodulation therapy into a neurophysiological signal set for each individual stimulation trial of the at least one stimulation trial is disclosed. The method includes subtracting, using a computing device, a trial-averaged signal from each individual stimulation trial of the at least one stimulation trial; interpolating, using the computing device, the series of signals within a baseline period and interpolating within a stimulation period containing the stimulation artifacts for each individual stimulation trial of the at least one stimulation trial; extracting, using the computing device, a non-phased-locked response from the series of signals for each individual stimulation trial of the at least one stimulation trial; subtracting, using the computing device, a baseline spectral response within the baseline period from a stimulation spectral response within the stimulation period to produce a neurophysiological signal set for each individual stimulation trial of the at least one stimulation trial; and displaying, using the computing device, the neurophysiological signal set for each individual stimulation trial of the at least one stimulation trial, wherein each neurophysiological signal set corresponds to each signal set of the series of signal sets with stimulation artifacts removed. Each neurophysiological signal set is indicative of the efficacy of the neuromodulation therapy. In some aspects, the method of interpolation of the baseline period and the method of interpolation of the stimulation period are independently selected from a linear interpolation method, a Crowther method, a polynomial curve fitting method, a matching pursuit method, and a wavelet interpolation method. In some aspects, the method preserves a temporal signal, a spectral signal, and a region-specific neural response within the measured neurophysiological signals during the electrical stimulation. In some aspects, the electrical stimulation is a constant-current square-wave delivered at an interval from 0.1 ms to 1 ms and a frequency from 0.5 Hz to 250 Hz.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic diagram of an existing method of interpolation-induced artifacts in bandpass-filtered neural signals. Ground truth signals (top, left) are contaminated with synthetic stimulation artifacts (top right). Linear interpolation is applied (bottom left), followed by bandpass filtering (70-170 Hz) and the Hilbert transform to extract the broadband high-frequency activity (BHA) envelope. This process introduces interpolation-related artifacts that distort true BHA responses (bottom right).

FIG. 1B is a schematic diagram showing that the disclosed method of stimulation artifact removal (also referred to herein as LIBRA) applies the same linear interpolation to both stimulation and baseline periods in one aspect of the method. Bandpass filtering and the Hilbert transform are then used to extract the BHA envelope, and subtraction of the baseline period removes interpolation-induced artifacts. In contrast, standard interpolation (see FIG. 1A) is applied only during stimulation and leaves large residual artifacts.

FIG. 1C is a step-by-step illustration of the disclosed method of stimulation artifact removal (also referred to herein as LIBRA).

FIG. 1D is a conceptual illustration comparing disclosed method of stimulation artifact removal (also referred to herein as LIBRA) with other artifact removal approaches. Unlike standard interpolation, template subtraction, and model decomposition-which are applied only during the stimulation period LIBRA employs a baseline-integrated approach to minimize interpolation-induced artifacts.

FIG. 2A is a schematic (top left) and corresponding time-frequency plots showing ground truth (top right), standard interpolation (bottom left), and the disclosed method of stimulation artifact removal (also referred to herein as LIBRA) (bottom right). Time-frequency plots from two representative SEEG contacts in response to auditory stimuli show that standard interpolation retains broadband spectral artifacts, whereas LIBRA effectively eliminates them after baseline subtraction. Contact C1 is an auditory-responsive electrode located in Heschl's gyrus, while contact C2 is a nonresponsive electrode located in the middle temporal cortex.

FIG. 2B is a set of images showing ground truth (left) and the disclosed method of stimulation artifact removal (also referred to herein as LIBRA) (right). LIBRA was validated using synthetic stimulation artifacts applied to ground truth intracranial signals recorded during a receptive language task. A 50 Hz synthetic stimulation during the auditory stimulus period (100-600 ms post-stimulus onset) with an 8 ms interpolation window was used for intuitive evaluation. Left panel: Topographic distribution of auditory-related broadband high-frequency activity (BHA) in ground truth signals. Dot size represents −log10(p) values comparing BHA during auditory stimulation vs. baseline. Data from electrocorticographic (ECoG) and stereoelectroencephalographic (SEEG) recordings are shown in the top and bottom rows, respectively. Right panel: Corresponding topographic distribution after LIBRA denoising, showing high overlap with ground truth. #Synthetic stim. Frequency: 50 Hz; interpolation duration: 8 ms.

FIG. 2C is a set of plots showing (top panel) the correlation of baseline-corrected BHA envelope (stimulus minus baseline) between ground truth and denoised signals. Each black dot represents the Pearson correlation value of an individual channel. Bottom panel: Correlation of BHA significance between ground truth and denoised signals. Each black dot represents the −log10(p) of an individual channel, where p is derived from one-sample two-tailed t-tests comparing baseline and stimulation periods. Data include all ECoG and SEEG channels.

FIG. 2D is a set of graphs showing mean power spectral density of auditory response (top left, n=62) and non-response (bottom left, n=117) channels during the stimulus (red) and baseline (black) periods. The right panel shows mean power spectral density of LIBRA-denoised signals for auditory response (top) and non-response (bottom). Auditory response channels were defined as those with p<10⁻⁵⁰, while non-response channels had p>0.95.

FIG. 2E is a set of graphs showing the mean power spectral density difference (stimulus minus baseline) for auditory response (top) and non-response (bottom) channels in ground truth (gray) and LIBRA-denoised (blue) signals. Auditory response channels were defined as those with p<10⁻⁵⁰, while non-response channels had p>0.95.

FIG. 3A is a set of plots where the disclosed method of stimulation artifact removal (also referred to herein as LIBRA) was compared with three alternative artifact removal methods: baseline-integrated physiological interpolation (BIPI), physiological-informed interpolation (PII), and standard linear interpolation (SLI). Performance under a representative synthetic stimulation condition (50 Hz stimulation frequency with an 8 ms interpolation duration). Correlation of baseline-corrected BHA envelope (stimulus minus baseline) between ground truth and denoised signals. Each black dot represents the Pearson correlation value of an individual channel.

FIG. 3B is a set of plots where the disclosed method of stimulation artifact removal (also referred to herein as LIBRA) was compared with three alternative artifact removal methods and performed under a representative synthetic stimulation condition (50 Hz stimulation frequency with an 8 ms interpolation duration). Correlation of BHA significance between ground truth and denoised signals. Each black dot represents the −log10(p) of an individual channel, where p is derived from one-sample two-tailed t-tests comparing baseline and stimulation periods. Data include all ECoG and SEEG channels.

FIG. 3C is a set of heatmaps comparing the disclosed method of stimulation artifact removal (also referred to herein as LIBRA) performance across six synthetic stimulation frequencies (10-130 Hz) and seven interpolation durations (2-30 ms). Top row: Pearson correlation (r) between BHA in the ground truth and denoised signals, quantifying the preservation of true neurophysiological activity. Middle row: Specificity (%), representing the proportion of non-response channels being correctly classified. Bottom row: Sensitivity (%), representing the proportion of response channels being correctly identified.

FIG. 3D is a set of graphs of mean (±s.d., n=31) correlation, specificity, and sensitivity across all synthetic stimulation conditions. the disclosed method of stimulation artifact removal (also referred to herein as LIBRA) achieved the highest correlation values, significantly outperforming BIPI (p=3.9×10−3), PII (p=1.2×10⁻⁸), and SLI (p=1.3×10⁻⁶). *p<0.01, one-sample two-tailed t-tests.

FIG. 4A is a set of representative BHA envelope traces (mean±s.d.) for auditory response channels (n=111) and non-response channels (n=117). The auditory stimulus starts at time zero, with synthetic stimulation applied at 300 ms post-stimulus onset. Signals are denoised with an 8 ms interpolation duration. the disclosed method of stimulation artifact removal (also referred to herein as LIBRA) is compared with baseline-integrated physiological interpolation (BIPI), physiological-informed interpolation (PII), and standard linear interpolation (SLI) under SPES.

FIG. 4B is a representative set of plots showing correlation between baseline-corrected BHA envelope (stimulus minus baseline) in ground truth and denoised signals during the 0-5 ms period after interpolation with an 8 ms interpolation duration. Each black dot represents the Pearson correlation value of an individual channel.

FIG. 4C is a graph of mean (±s.d., n=35) correlation values across seven interpolation durations (2-30 ms) and five post-interpolation time periods (0-25 ms). the disclosed method of stimulation artifact removal (also referred to herein as LIBRA) demonstrated significantly higher correlation values compared to BIPI (p=1.7×10⁻³), PII (p=9.5×10−5), and SLI (p=1.5×10−3). *p<0.01, one-sample two-tailed t-tests.

FIG. 4D is a set of heatmaps detailing the disclosed method of stimulation artifact removal (also referred to herein as LIBRA) performance across conditions. Pearson correlation (r) between the ground truth and denoised BHA signals is shown across different methods.

FIG. 5A is a set of images showing spatial distribution of baseline-corrected BHA correlations with stimulation intensity during 50 Hz stimulation in the lateral (top) and basal (bottom) amygdala. Lateral amygdala stimulation led to positive BHA correlations in the orbitofrontal cortex (OFC) and inferior frontal gyrus (IFG), while basal amygdala stimulation resulted in negative BHA correlations in the anterior cingulate cortex (ACC) and superior frontal gyrus (SFG). Significant positive (red) and negative (blue) correlations were determined using a randomization test (p<0.01).

FIG. 5B is a set of graphs showing the number of SEEG channels showing significant correlations between BHA and stimulation intensity across frontal lobe regions during lateral (top) and basal (bottom) amygdala stimulation (p <0.01, randomization test).

FIG. 5C is a set of graphs of mean (±s.e.m.) power spectral density of significant channels in OFC/IFG (lateral amygdala stimulation, red, n=14) and ACC/SFG (basal amygdala stimulation, blue, n=22). Power is shown during the baseline (gray) and stimulation (black) periods. Linear interpolation was applied to both periods.

FIG. 5D is a set of graphs of dose-dependent mean (±sd) baseline-corrected BHA across significant OFC/IFG channels during lateral amygdala stimulation (n=14) and ACC/SFG channels during basal amygdala stimulation (n=22).

FIG. 5E is a set of plots of mean cortico-cortical evoked potential (top; CCEPs; green) for OFC/IFG channels during lateral amygdala stimulation (left, n =14) and ACC/SFG channels during basal amygdala stimulation (right, n=22) and mean baseline-corrected BHA envelope (bottom; green) for OFC/IFG channels during lateral amygdala stimulation (left, n=14; p=2.3×10−4) and ACC/SFG channels during basal amygdala stimulation (right, n=22, p=1.5×10−7). *p<0.01, one-sample two-tailed t-tests. Individual channel responses are shown in black.

FIG. 6A is a schematic illustration of linear and physiological interpolation during stimulation (left) and baseline (right) periods. In linear interpolation, artifact-affected segments are replaced by a straight-line fit between the surrounding signals. In physiological interpolation, artifact segments are reconstructed by merging reversed, tapered signals from both sides of the artifact window. Black traces represent raw signals, green traces represent physiological components used in reconstruction, and red traces represent the final interpolated signal. In physiological interpolation, the red trace results from the pointwise summation of the two green traces.

FIG. 6B is an overview showing the disclosed method of stimulation artifact removal (also referred to herein as LIBRA) compared with three established artifact removal methods: standard linear interpolation (SLI), physiological-informed interpolation (PII), and baseline-integrated physiological interpolation (BIPI). LIBRA applies linear interpolation to both baseline and stimulation periods and subtracts the two to isolate true neurophysiological responses while minimizing interpolation-induced artifacts. SLI replaces artifact segments only during the stimulation period with a linear interpolation. PII reconstructs artifact segments by merging surrounding physiological signals. BIPI extends PII by applying the same interpolation to both baseline and stimulation periods, serving as a closer control for LIBRA's baseline-integrated approach.

FIG. 7A is a set of representative graphs showing the correlation between baseline-corrected BHA responses of ground truth and denoised signals under 50 Hz synthetic stimulation across five interpolation durations (2-10 ms). Higher Pearson correlation values indicate better preservation of ground truth signals. Each black dot represents one ECoG or SEEG channel.

FIG. 7B is a set of representative graphs showing the correlation of BHA significance between ground truth and denoised signals across interpolation durations. Specificity (sp, blue) and sensitivity (sn, blue) values indicate the proportion of correctly classified non-response and response channels, respectively. Each black dot represents the −log10(p) of an individual channel, where p is derived from one-sample two-tailed t-tests comparing baseline and stimulation periods.

FIG. 7C is a set of representative graphs showing the correlation between baseline-corrected BHA responses of ground truth and denoised signals under single pulse synthetic stimulation, evaluated over five post-interpolation periods (0-25 ms) for a representative 8 ms interpolation duration. Each black dot represents the Pearson correlation of a single channel.

FIG. 8 is a set of spectral envelope graphs where averaged (mean±s.d., n=31) correlation, specificity, and sensitivity across synthetic stimulation frequencies (10-130 Hz) and interpolation durations (2-30 ms) are shown for the spectral envelope in six frequency bands: theta, alpha, low beta, high beta, low gamma, and BHA. The correlation metric represents the relationship between neural responses of denoised signals and the ground truth signal, with higher values indicating better preservation of true neurophysiological activity. Sensitivity reflects the proportion of ground truth response channels correctly identified by the artifact removal methodologies, while specificity represents the proportion of non-response channels correctly classified as non-responsive. the disclosed method of stimulation artifact removal (also referred to herein as LIBRA) is compared with three established artifact removal methods: SLI, PII, and BIPI. LIBRA demonstrates similar performance to other methodologies in lower frequency bands (e.g., theta and alpha) but significantly outperforms them in higher frequency bands, particularly low gamma and BHA. Statistical comparisons between LIBRA and the other methodologies show p-values as follows: theta (p=1.5×10⁻¹, p=1.7×10⁻¹, p=2.2×10⁻¹), alpha (p=7.6×10⁻², p=1.1×10⁻², p=2.7×10⁻²), low beta (p=1.3×10⁻¹, p=4.8×10⁻³, p=1.4 ×10⁻²), high beta (p=2.4×10⁻², p=2.3×10⁻⁴, p=1.4×10⁻³), low gamma (p=8.0×10⁻³, p=1.6×10⁻⁷, p=1.1×10⁻⁵), and BHA (p=3.9×10⁻³, p=1.2×10⁻⁸, p=1.3×10⁻⁶) when compared with BIPI, PII, and SLI, respectively. *p<0.01; ns: p≥0.01; one-sample two-tailed t-tests.

FIG. 9A is a diagram showing representative amygdala nuclei and intracranial stereoelectroencephalographic (SEEG) contacts implanted in the amygdala for stimulation and recording.

FIG. 9B is a set of plots of mean (±s.d.; n=204) absolute neural recordings across channels during lateral amygdala stimulation (left panel) and basal amygdala stimulation (right panel). The grey traces represent neural recordings from individual channels. Time zero corresponds to the onset of each stimulation pulse. Dashed lines indicate the time period used for linear interpolation (3 ms before stimulation onset and 5 ms after stimulation onset) in the analysis.

FIG. 9C is a representation set of plots of averaged (mean±s.e.m., n=10) raw neural recordings in the OFC/IFG and ACC/SFG regions during 50 Hz electrical stimulation (3 mA) in the lateral and basal amygdala, highlighting the stimulation artifacts and gamma entrainment observed in the orbitofrontal cortex (OFC), inferior frontal gyrus (IFG), anterior cingulate cortex (ACC), and superior frontal gyrus (SFG).

FIG. 10A is a representative set of brain images showing the correlation between BHA and stimulation intensities in non-overlapping 250 ms time bins during the stimulation period (0-1000 ms after stimulation onset) and poststimulation period (1000-2000 ms after stimulation onset) with lateral amygdala stimulation. Red and blue circles represent channels with significant (p<0.05) positive and negative correlations, respectively.

FIG. 10B is a representative set of brain images showing the correlation between BHA and stimulation intensities in non-overlapping 250 ms time bins during the stimulation period (0-1000 ms after stimulation onset) and poststimulation period (1000-2000 ms after stimulation onset) with basal amygdala stimulation. Red and blue circles represent channels with significant (p<0.05) positive and negative correlations, respectively. For lateral amygdala stimulation, the p-values for each time bin from 0 to 2000 ms are: p=1.4×10⁻³, p=3.3×10⁻¹¹, p=2.0×10⁻¹⁰, p=9.0×10⁻¹⁰, p=3.3×10⁻², p=1.7×10⁻¹, p=4.9×10⁻¹, p=1.6×10^{-2.}

FIG. 10C is a set of plots showing mean (±s.d.) correlation of BHA with stimulation intensities across different time bins in the OFC/IFG regions during lateral amygdala stimulation (top, n=14) and in the ACC/SFG regions during basal amygdala stimulation (bottom, n=22). Significant time bins are marked with *p<0.05; ns: p≥0.05 (one-sample two-tailed t-tests). For basal amygdala stimulation, the p-values for each time bin from 0 to 2000 ms are: p=5.2×10⁻⁹, p=1.0×10⁻¹⁶, p=6.7×10⁻¹¹, p=3.0×10⁻¹⁴, p=6.8×10⁻¹⁰, p=8.1×10⁻¹, p=1.3×10⁻⁴, p=1.4×10⁻⁴. OFC: orbitofrontal cortex; IFG: inferior frontal gyrus; ACC: anterior cingulate cortex; SFG: superior frontal gyrus.

FIG. 11A is a set of graphs comparing power spectral density (PSD) during baseline (black) and stimulation (red) periods for ground truth signal (left), traditional denoising method (middle), and the disclosed method of stimulation artifact removal (also referred to herein as LIBRA), a baseline-integrated denoising method (right).

FIG. 11B is a graph demonstrating the disclosed method of stimulation artifact removal (also referred to herein as LIBRA) effectively removing interpolation-induced artifacts by leveraging congruency between the baseline and stimulation periods. Baseline-corrected PSD (mean±s.e.m. ; n=100) is shown for the ground truth signal (black), the denoised signal using traditional methods (blue), and the denoised signal using LIBRA (green). LIBRA achieves a close overlap with the ground truth, whereas traditional methods exhibit substantial deviations.

FIG. 12A is an illustration comparing the disclosed method of stimulation artifact removal (also referred to herein as LIBRA) with existing artifact removal methods, interpolation, template subtraction, model decomposition, and LIBRA. LIBRA introduces a baseline-integrated approach that enhances artifact removal while preserving neurophysiological responses.

FIG. 12B is a set of graphs showing high-frequency electrical stimulation (HFES; top) and single-pulse electrical stimulation (SPES; bottom). the disclosed method of stimulation artifact removal (also referred to herein as LIBRA) removes the artifacts by replacing artifact-affected segments with linearly interpolated values (green) and applying the same interpolation parameters to the baseline.

FIG. 13 is a set of images showing broadband high-frequency activity (BHA) recorded from the human frontal lobe with basal and lateral amygdala electrical stimulation. Spatial distribution of baseline-corrected BHA correlation with stimulation intensities during 50 Hz electrical stimulation in the lateral (top) and basal (bottom) amygdala. Lateral amygdala stimulation elicited positive (red) BHA correlations in orbitofrontal cortex (OFC) and inferior frontal gyrus (IFG) regions, whereas basal amygdala stimulation elicited negative (blue) BHA correlations in anterior cingulate cortex (ACC) and superior frontal gyrus (SFG) regions. Red and blue circles indicate channels with significant (p<0.01, randomization test) positive and negative correlations, respectively. The coronal (left), sagittal (middle), and horizontal (right) planes are shown.

FIG. 14A is a set of graphs of broadband high-frequency activity (BHA) under different stimulation intensities. Mean BHA (±s.e.m. ; n=14 for orbitofrontal cortex/inferior frontal gyrus [OFC/IFG], n=22 for anterior cingulate cortex/superior frontal gyrus [ACC/SFG]) during lateral and basal amygdala 50 Hz stimulation at varying stimulation intensities (0.5, 1, 2, and 3 mA). Linear interpolation was applied to the baseline period (1100 ms to 100 ms before stimulation onset) and the stimulation period (0 ms to 1000 ms after stimulation onset). Black traces represent the average across all stimulation intensities, showing clear interpolation-related artifacts in both baseline and stimulation periods.

FIG. 14B is a set of graphs of baseline-corrected BHA (mean±s.e.m.) for each stimulation intensity, calculated by subtracting baseline BHA from stimulation-period BHA. From left to right: Lateral amygdala stimulation (OFC/IFG), lateral amygdala stimulation (ACC/SFG), basal amygdala stimulation (OFC/IFG), and basal amygdala stimulation (ACC/SFG).

FIG. 14C is a set of graphs depicting mean baseline-corrected BHA (±s.d. ; n=14 for OFC/IFG, n=22 for ACC/SFG) as a function of stimulation intensity. Correlation analysis (Pearson correlation) revealed strong positive correlations between baseline-corrected BHA and stimulation intensity for lateral amygdala stimulation in OFC/IFG (r=0.74, p=5.4×10⁻¹¹) and a weaker correlation in ACC/SFG (r=0.15, p=1.7×10⁻¹). Basal amygdala stimulation induced strong negative correlations in ACC/SFG (r=0.75, p=4.0×10⁻¹⁷) and moderate positive correlations in OFC/IFG (r=0.59, p=1.6×10⁻⁶). OFC: orbitofrontal cortex; IFG: inferior frontal gyrus; ACC: anterior cingulate cortex; SFG: superior frontal gyrus.

FIG. 15 is a block diagram schematically illustrating a stimulation artifact removal system in accordance with one aspect of the disclosure.

FIG. 16 is a block diagram schematically illustrating a computing device in accordance with one aspect of the disclosure.

FIG. 17 is a block diagram schematically illustrating a remote or user computing device in accordance with one aspect of the disclosure.

FIG. 18 is a block diagram schematically illustrating a server system in accordance with one aspect of the disclosure.

Those of skill in the art will understand that the drawings described herein are for illustrative purposes only. The drawings are not intended to limit the scope of the present teachings in any way.

### DETAILED DESCRIPTION

The present disclosure is based, at least in part, on the discovery of a method to remove electrical stimulation artifacts in electrophysiology data with Linear Baseline-integrated Removal of Artifacts (LIBRA).

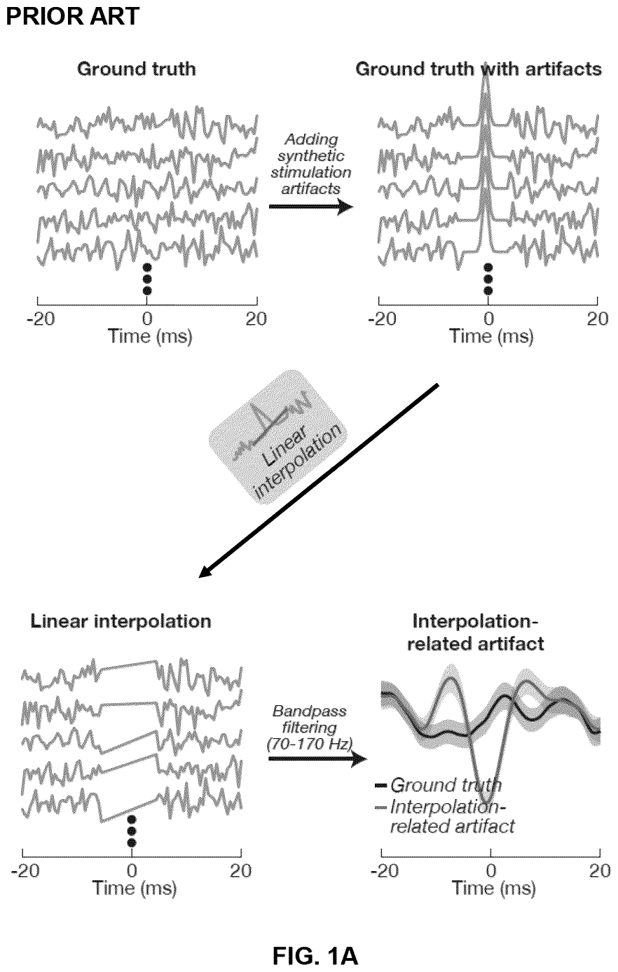

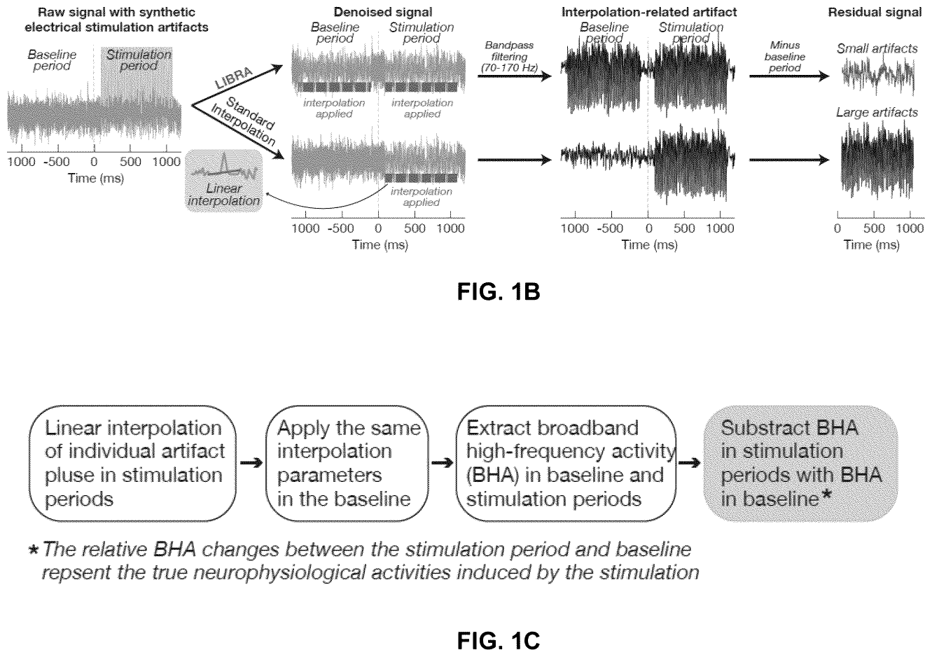

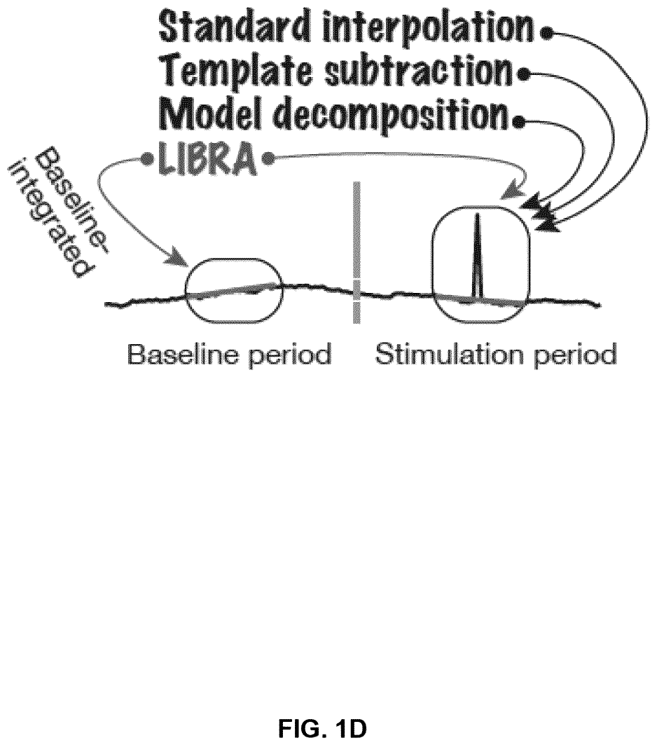

Without being limited to any particular theory, extracting broadband high-frequency activity (BHA; 70-170 Hz) during electrical stimulation presents a major technical challenge due to residual artifacts that can distort true neurophysiological signals even after applying standard artifact removal methods (FIG. 1D). In particular, bandpass filtering, commonly used to isolate BHA, exacerbates the temporal spread of residual artifacts due to the uncertainty principle, resulting in spectral leakage that mimics genuine neural activity.

In various aspects, the disclosed method of stimulation artifact removal (also referred to herein as LIBRA) removes high-frequency artifacts from electrophysiology data, making it possible to study the neural effects during a stimulation period. Traditional technologies and methods primarily attempt to remove stimulation artifacts only during the stimulation period and will introduce new artifacts to the data. The LIBRA approach overcomes these limitations by performing interpolation on both non-stimulation (thus, it is “baseline-integrated”) and stimulation periods, thus maintaining relative changes in the data while eliminating the artifacts generated by LIBRA itself.

In one aspect, a method of stimulation artifact removal (also referred to herein as LIBRA, a LInear Baseline-integrated Removal of Artifacts), a methodology designed to effectively remove stimulation artifacts while preserving neurophysiological BHA activity during both single-pulse electrical stimulation (SPES) and high-frequency electrical stimulation (HFES) is disclosed and shown illustrated in FIG. 1B and FIG. 1C. LIBRA represents a framework for artifact removal, shifting the focus from traditional methods that analyze only stimulation-period data to a baseline integrated approach (FIG. 1D). Furthermore, unlike existing techniques that rely on complex modeling or decomposition methods, LIBRA employs a simple, yet effective linear interpolation strategy applied consistently across both baseline and stimulation periods. This simplicity makes LIBRA easy to implement while providing robust and reliable performance, offering a transformative solution for analyzing neural activity during electrical stimulation.

Without being limited to any particular theory, an artifact-affected signal obtained during electrical stimulation typically includes three main components: (1) stimulation induced artifacts, including a transient, high-amplitude waveform resembling a Dirac function and a slower capacitive discharge; (2) phase-locked neurophysiological responses, such as cortico-cortical evoked potentials from SPES and oscillatory entrainment from HFES; and (3) non-phase-locked neurophysiological responses, such as BHA12, which reflect ongoing neural activity and are typically the primary signals of interest.

Without being limited to any particular theory, effective BHA extraction requires removing both stimulation-induced artifacts and contamination from phase-locked responses, which may obscure non-phase-locked BHA. To address this challenge, LIBRA (LInear Baseline integrated Removal of Artifacts) implements a two-stage denoising strategy. In the first stage, slower capacitive discharge and phase-locked responses are removed using trial-averaging (template subtraction), a method previously shown to be effective. However, residual transient artifacts often persist due to trial-to-trial variability. In the second stage (FIG. 1C), LIBRA replaces these transient artifacts with linear interpolation and applies the same interpolation parameters to a baseline period, ensuring that interpolation-induced spectral artifacts are matched across baseline and stimulation periods. BHA is then extracted separately from each period, and subtraction of the baseline from the stimulation response isolates true BHA while eliminating shared interpolation-induced artifacts.

In some aspects, the LIBRA denoising method includes five steps.
Step-I: Subtract trial-averaged signals. Remove slower capacitive discharge and phase-locked neurophysiological responses by subtracting the trial-averaged signal from individual stimulation trials.
Step-II: Linear interpolation of transient stimulation artifact. Replace the transient artifact component of each stimulation pulse using linear interpolation.
Step-III: Baseline-integrated interpolation. Apply the same interpolation parameters to the baseline period to ensure consistency in interpolation-induced artifacts.
Step-IV: Extract BHA envelope. Extract the BHA envelope separately for the baseline and stimulation periods using bandpass filtering (e.g., finite impulse response filter) followed by the Hilbert transform.
Step-V: Baseline subtraction. Subtract the BHA envelope in the baseline period from those in the stimulation period. The relative changes (i.e., baseline corrected response) are assumed to represent true neurophysiological BHA responses induced by electrical stimulation.

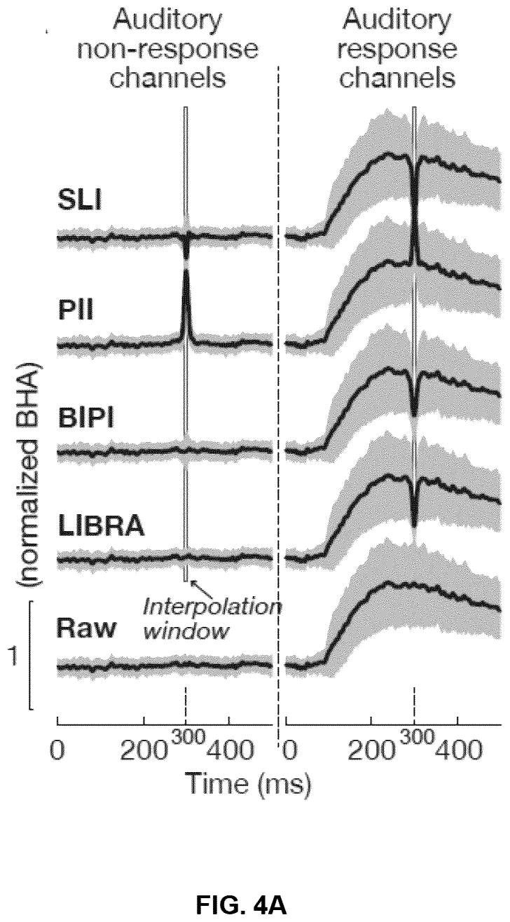

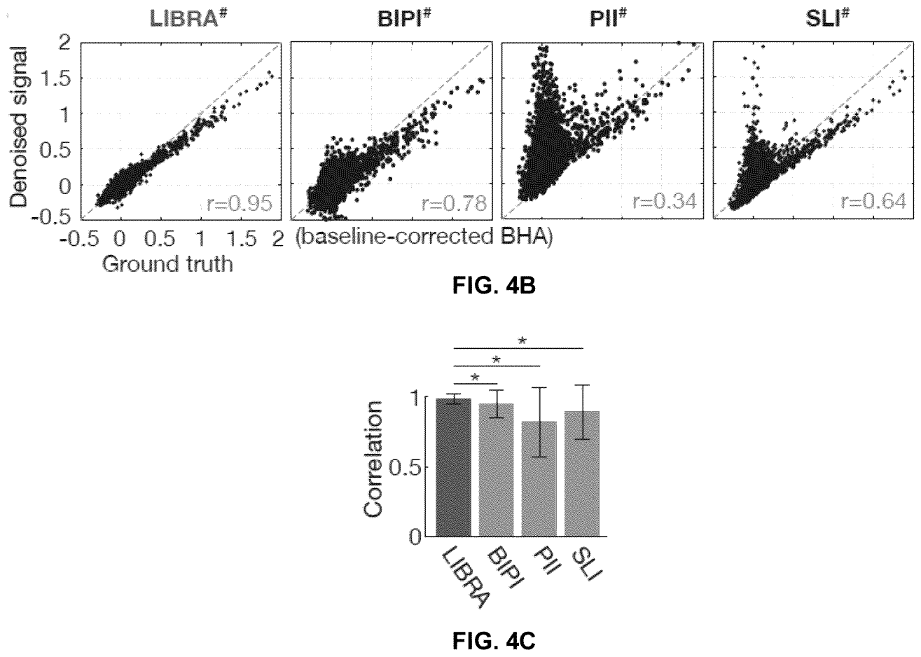

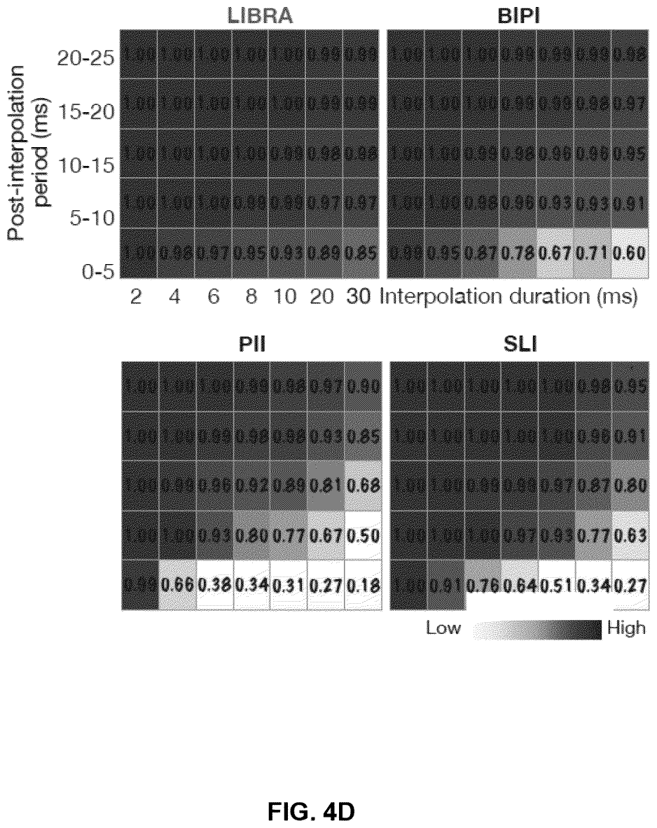

In other aspects, the disclosed artifact removal method includes initially identifying individual stimulation artifact pulses in electrophysiological data. The parameters used to identify these pulses include the width of each pulse, the frequency of pulses, and the duration of stimulation trains. The pulses are then detected based on an onset time, and a segment of data containing the pulse (as determined by the width of each pulse) is deleted and replaced by interpolated signal values determined by a user-specified interpolation method. The user-specified interpolation methods that can be used include but are not limited to linear interpolation, Crowther method, polynomial curve fitting methods (includes multiple polynomial orders), matching pursuit method, and wavelet interpolation methods. In some aspects, Additional interpolation methods may be used, including adaptive approaches that can account for any variability in interpolation-induced artifacts between the baseline and stimulation periods. This provides the user with the flexibility to select the most relevant interpolation method given the quality of the data collected, or the nature of the artifact. The same interpolation parameters used during the stimulation period are also applied to the baseline period, thus ensuring consistency between the stimulation and baseline periods before downstream analysis. Compared to other common high-frequency artifact removal methods, LIBRA demonstrates superior conservation of physiological effects (FIG. 4A, FIG. 4B).

The present method provides electrophysiology researchers with a valuable tool to overcome a prevalent data analysis problem: preserving true physiological effects in the presence of high frequency artifacts. The baseline-integrated approach leveraged by LIBRA ensures that any artificial noise introduced during artifact removal is consistent across the baseline and stimulation periods, enabling the conservation of electrophysiological activities during stimulation.

LIBRA provides a framework for artifact removal, shifting the focus from traditional methods that analyze only stimulation-period data to a baseline-integrated approach (FIG. 1D). By leveraging baseline subtraction to account for interpolation-induced artifacts, LIBRA ensures accurate extraction of neural responses while introducing this methodological perspective. Despite its innovative nature, LIBRA is remarkably simple in its design, relying on straightforward linear interpolation. This simplicity makes it computationally efficient and easy to implement without requiring sophisticated training. As a result, LIBRA can be widely adopted by researchers across disciplines, providing reliable results and facilitating broader access to advanced neurophysiological analyses during electrical stimulation.

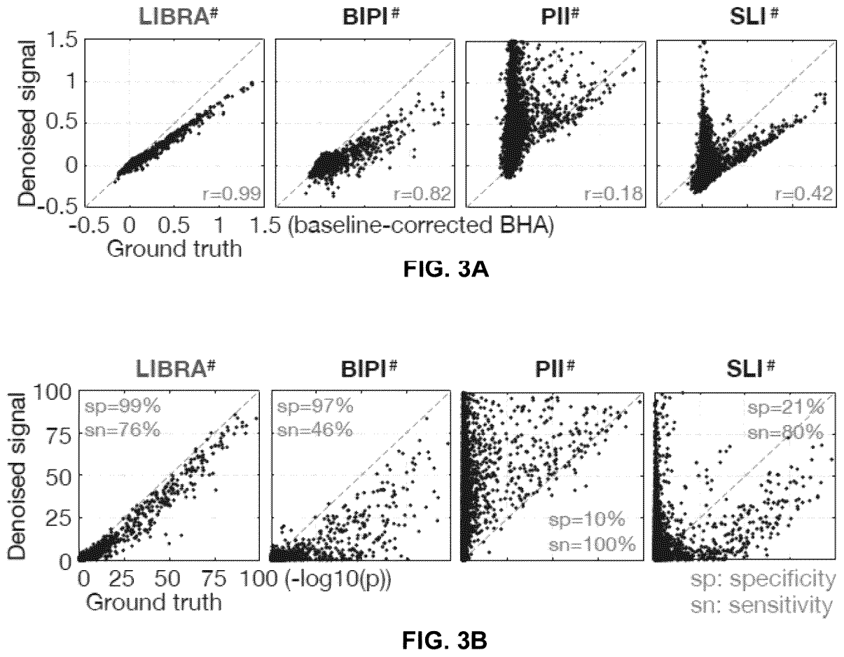

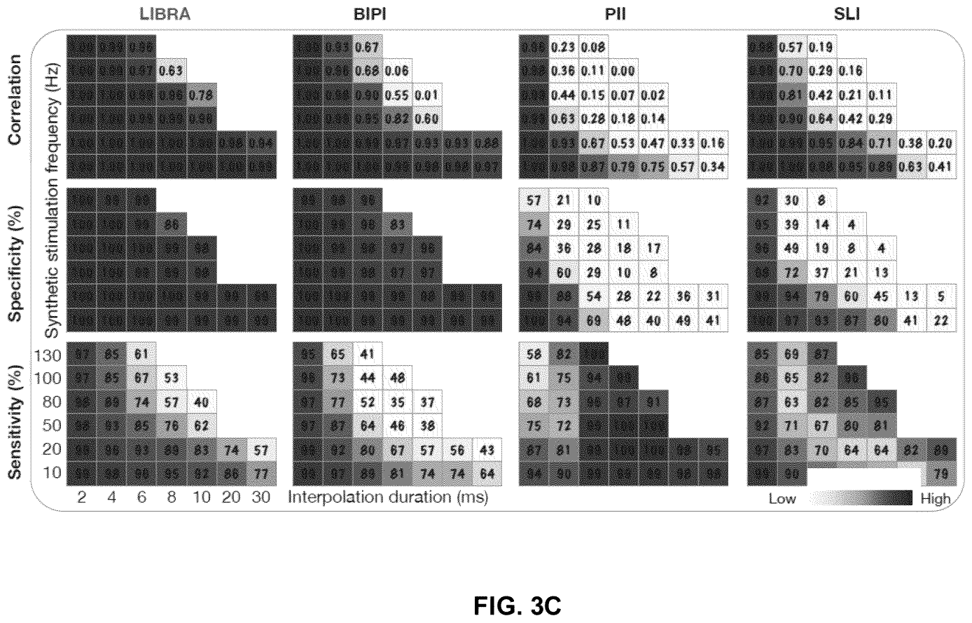

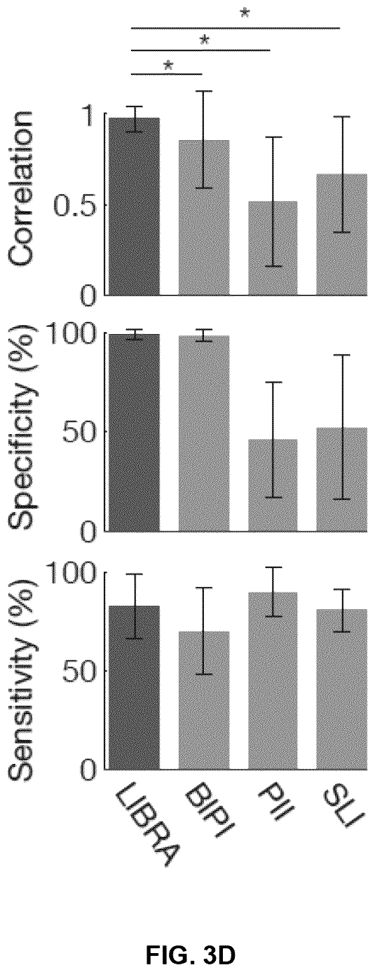

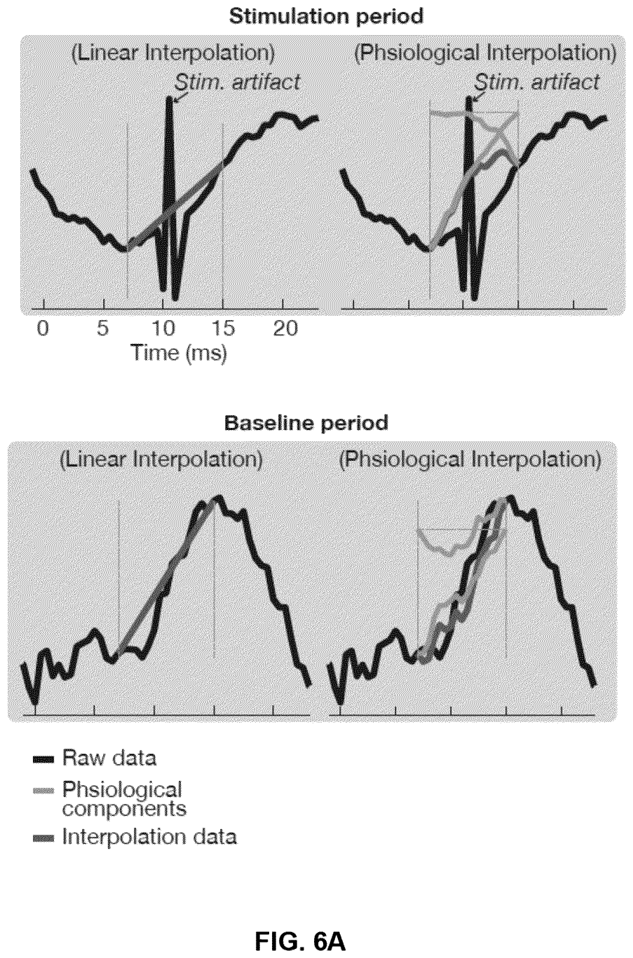

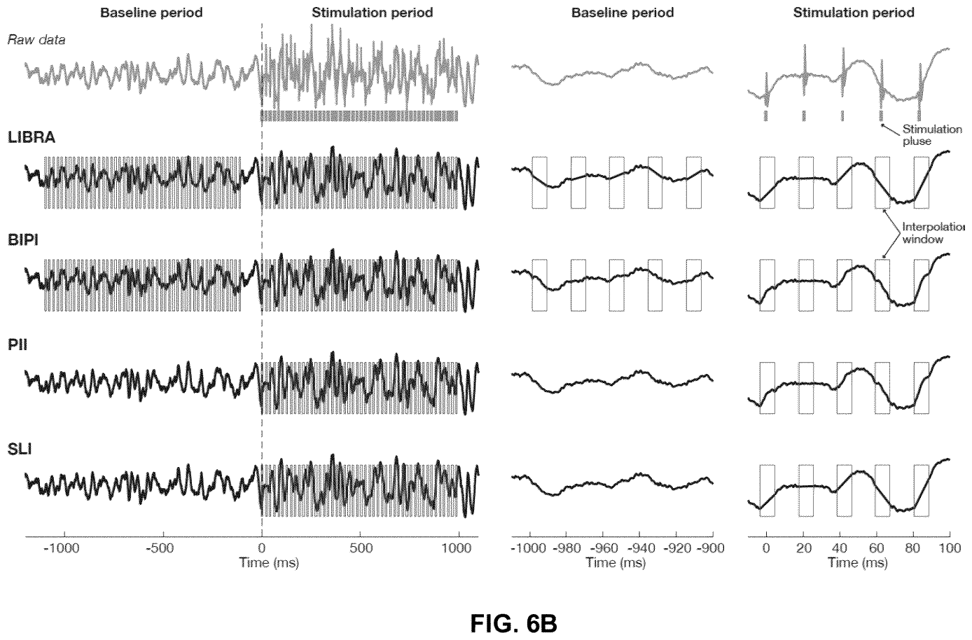

Without being limited to any particular theory, the effectiveness of LIBRA artifact removal methods relies on the assumption that interpolation-induced artifacts in the baseline period are consistent with those in the stimulation period. This assumption is supported by two key factors. First, the baseline and stimulation periods are temporally proximate, ensuring relatively stationary neural dynamics over short intervals (e.g., within a few milliseconds). Second, LIBRA's use of simple linear interpolation ensures that any artifacts introduced remain consistent across both baseline and stimulation periods. To evaluate this, LIBRA was compared with BIPI, which also employs a baseline-integrated strategy but reconstructs artifact-affected segments by merging surrounding neurophysiological signals rather than using linear interpolation. The physiological interpolation approach unintentionally increases signal variability between baseline and stimulation periods, while the consistent artifact processing of LIBRA's linear interpolation minimizes such variability (FIG. 6A). This distinction underscores LIBRA's enhanced ability to preserve true neural responses compared to existing methods such as BIPI (FIG. 3D).

### Electrical Brain Stimulation

Electrical brain stimulation may be used to modulate neural activity, in neurosciences, neuroengineering research, and for clinical treatments for neurological and psychiatric disorders. Electrical brain stimulation may use broadband high-frequency activity (BHA) at 70-170 Hz. Other frequencies may include theta, alpha, low beta, high beta, and low gamma.

Electrical stimulation may be delivered as 0.1-1 ms constant-current square-wave pulses at rates ranging from 0.5-250 Hz. Each pulse generates large stimulation artifacts characterized by transient morphology with a broad spectral power increase followed by a slower capacitive discharge. An artifact-affected signal typically includes stimulation-induced artifacts (the transient stimulation artifact and the slower capacitive discharge) and accompanying neurophysiological responses, which are categorized as phase-locked and non-phase-locked components. Phase-locked responses, which are time-locked to the stimulation onset, can be reliably extracted by averaging signals across trials in the temporal domain. As stimulation artifacts are mainly confined to a narrow window of a few milliseconds, phase-locked responses typically occur outside this window and are minimally affected by artifacts.

Non-phase-locked responses are observed in the spectral domain and reflect broad-band high-frequency activity (BHA) as well as other spectral components within canonical frequency bands. Bandpass filtering is a common approach for extracting these responses. However, due to the uncertainty principle, residual artifacts can spread temporally when transformed into the spectral domain.

### Computing Systems

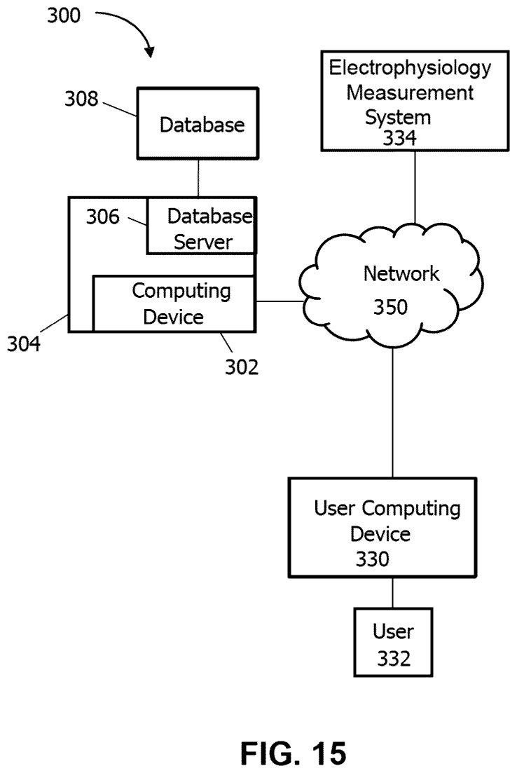

In various aspects, the disclosed methods may be implemented using a computing system or computing device. FIG. 15 depicts a simplified block diagram of the system for implementing the computer-aided method described herein. As illustrated in FIG. 15, the computing device 300 may be configured to implement at least a portion of the tasks associated with the disclosed methods of removing stimulation artifacts from electrophysiology signals as described herein. The computer system 300 may include a computing device 302. In one aspect, the computing device 302 is part of a server system 304, which also includes a database server 306. The computing device 302 is in communication with a database 308 through the database server 306. The computing device 302 is communicably coupled to a user computing device 330 and an electrophysiology measurement system 334 through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

In other aspects, the computing device 302 is configured to perform a plurality of tasks associated with the disclosed computer-aided methods of treatment plan production and optimization. In some aspects, the computing device 302, user computing device 330, and/or electrophysiology measurement system 334 may be operatively connected via a network 350.

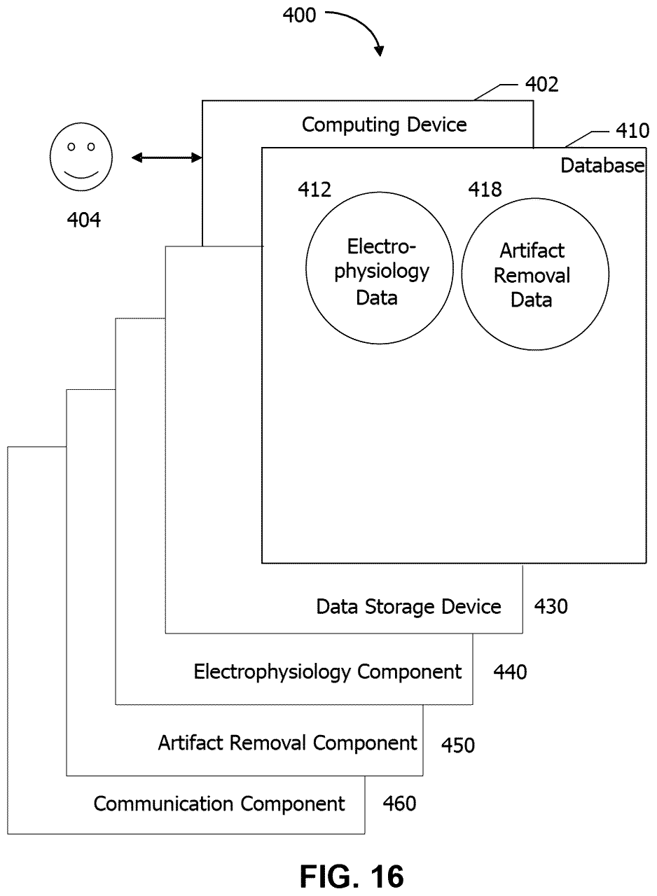

FIG. 16 depicts a component configuration 400 of computing device 402, which includes database 410 along with other related computing components. In some aspects, computing device 402 is similar to computing device 302 (shown in FIG. 15). A user 404 may access components of computing device 402. In some aspects, database 410 is similar to database 308 (shown in FIG. 15).

In one aspect, database 410 includes electrophysiology data 412 and artifact removal data 418. Non-limiting examples of electrophysiology data 412 includes signals indicative of electroencephalographic activity (EEG), electrocorticographic (ECoG) activity, stereo-electroencephalographic (SEEG) activity, magnetoencephalographic (MEG) activity, and local field potential (LFP) activity to be processed to remove stimulation artifacts using the methods as disclosed herein. Artifact removal data 418 may include any parameters defining results, equations, physical parameters, equations and other parameters defining the algorithms used to implement the artifact removal method as disclosed herein.

Computing device 402 also includes a number of components that perform specific tasks. In the exemplary aspect, computing device 402 includes a data storage device 430, an electrophysiology component 440, an artifact removal component 450, and a communication component 460. The electrophysiology component 440 is configured to operate device configured to measure signals indicative of electrophysiology as described herein. The artifact removal component 450 is configured to process the signals indicative of electrophysiology to remove stimulation artifacts using the method as described herein. The data storage device 430 is configured to store data received or generated by computing device 402, such as any of the data stored in database 410 or any outputs of processes implemented by any component of computing device 402.

The communication component 460 is configured to enable communications between computing device 402 and other devices (e.g. user computing device 330 shown in FIG. 15) over a network, such as a network 350 (shown in FIG. 15), or a plurality of network connections using predefined network protocols such as TCP/IP (Transmission Control Protocol/Internet Protocol).

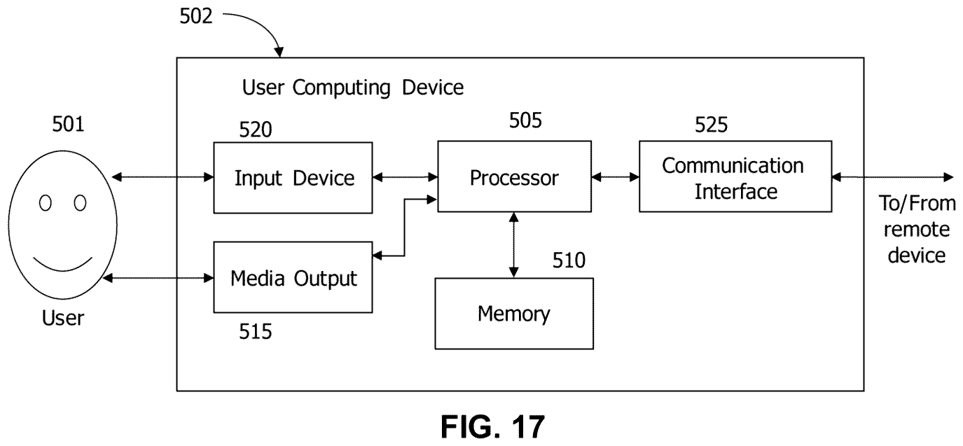

FIG. 17 depicts a configuration of a remote or user computing device 502, such as user computing device 330 (shown in FIG. 15). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light-emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

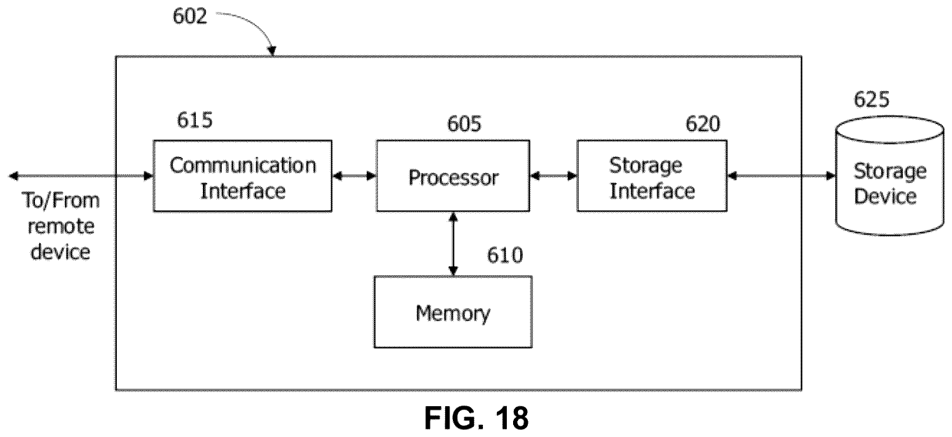

FIG. 18 illustrates an example configuration of a server system 602. Server system 602 may include, but is not limited to, database server 306 and computing device 302 (both shown in FIG. 15). In some aspects, server system 602 is similar to server system 304 (shown in FIG. 15). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 15) or another server system 602. For example, communication interface 615 may receive requests from a user computing device 330 via a network 350 (shown in FIG. 15).

Processor 605 may also be operatively coupled to a storage device 625. Storage device 625 may be any computer-operated hardware suitable for storing and/or retrieving data. In some aspects, storage device 625 may be integrated into server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIGS. 16) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only and are thus not limiting as to the types of memory usable for the storage of a computer program.

The computer systems and computer-aided methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

The methods and algorithms of the disclosure may be enclosed in a controller or processor. Furthermore, methods and algorithms of the present disclosure, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and backup drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods.

The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to images or frames of a video, object characteristics, and object categorizations. Data inputs may further include sensor data, image data, video data, telematics data, authentication data, authorization data, security data, mobile device data, geolocation information, transaction data, personal identification data, financial data, usage data, weather pattern data, “big data” sets, and/or user preference data. ML outputs may include but are not limited to: a tracked shape output, categorization of an object, categorization of a region within a medical image (segmentation), categorization of a type of motion, a diagnosis based on the motion of an object, motion analysis of an object, and trained model parameters ML outputs may further include: speech recognition, image or video recognition, medical diagnoses, statistical or financial models, autonomous vehicle decision-making models, robotics and animal behavior modeling, fraud detection analysis, user recommendations and personalization, game AI, skill acquisition, targeted marketing, big data visualization, weather forecasting, and/or information extracted about a computer device, a user, a home, a vehicle, or a party of a transaction. In some aspects, data inputs may include certain ML outputs.

In some aspects, at least one of a plurality of ML methods and algorithms may be applied, which may include but are not limited to: linear or logistic regressions, random forest classifiers, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, adversarial learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship.

In yet another aspect, ML methods and algorithms are directed toward reinforcement learning, which involves optimizing outputs based on feedback from a reward signal. Specifically, ML methods and algorithms directed toward reinforcement learning may receive a user-defined reward signal definition, receive a data input, utilize a decision-making model to generate an ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, an ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

### Definitions

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

As used herein, the term “neural signal” refers broadly to any signal reflecting the electromagnetic (EM) activity of the brain, without limitation. Non-limiting examples of neural signals include signals indicative of electroencephalographic activity (EEG), electrocorticographic (ECoG) activity, stereo-electroencephalographic (SEEG) activity, magnetoencephalographic (MEG) activity, and local field potential (LFP) activity.

As used herein, the term “neural targets” refers broadly to any brain site that is chosen as the target of brain stimulation.

As used herein, the term “neural response” refers broadly to the neural signal that is related (or response) to the brain stimulation.

As used herein, the term “stimulation response” refers broadly to the neural signal that is related to the brain stimulation.

As used herein, the term “stimulation artifacts” refers broadly to the non-neural signal that is not originally from neural activity, but instead, from the stimulation.

As used herein, the term “neuromodulation therapy” refers broadly to a range of medical procedures that use electrical stimulation or chemical agents to alter nerve activity and treat neurological and psychiatric disorders.

In various aspects, the disclosed method can be used to remove stimulation artifacts from different types of stimulation techniques: including repetitive transcranial magnetic stimulation (rTMS), single-pulse TMS, transcranial direct current stimulation (tDCS), transcranial random noise stimulation (tRNS), transcranial alternating current stimulation (tACS), transcranial focused ultrasound stimulation (tFUS), vagus nerve stimulation (VNS); single-pulse electrical stimulation (SEPS), high-frequency stimulation, and micro-stimulation

In various aspects, the disclosed method can be used to remove stimulation artifacts of different neural signals, including any signal reflecting the electromagnetic (EM) activity of the brain without limitation including, but not limited to, electroencephalographic activity (EEG), electrocorticographic (ECoG) activity, stereo-electroencephalographic (SEEG) activity, magnetoencephalographic (MEG) activity, and local field potential (LFP) activity.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Validation of Libra Method of Removal of Artifacts in Measurements of Electrophysiological Activity Induced During Single-Pulse and High Frequency Electrical Stimulation

To validate the disclosed method to remove stimulation artifacts from brain activity signals generated during electrical brain stimulation (Linear Baseline-Integrated Removal of Artifacts (LIBRA)), the following experiments were performed.

To validate LIBRA's performance, human intracranial neural recordings from a receptive language task were used as ground truth signals. Synthetic stimulation artifacts were introduced based on predefined electrical stimulation parameters, such as frequency and pulse width. The primary evaluation metric was the correlation between the BHA envelope in the denoised and ground truth signals, assessing LIBRA's ability to preserve true neurophysiological BHA while removing artifacts.

Intracranial signals were recorded from 25 patients (15 males, 10 females; age range 24-62 year; mean±s.d.: 36±9 years) undergoing epilepsy monitoring with electrocorticography (ECoG, n=6) or stereoelectroencephalography (SEEG, n=19). ECoG electrodes had 4 mm diameter contacts (3 mm exposed surface, 6-10 mm spacing; PMT Corp.), while SEEG electrodes had 0.8 mm diameter contacts (2 mm length, 1.5 mm insulation; PMT Corp.). Data were recorded at 1200 Hz (g.Hlamp, g. tec) for ECoG, and 2000 Hz (Nihon Kohden JE-120) for SEEG. The general-purpose BCI2000 software was used to record and save data for further analysis. Electrode locations were determined using preoperative MRI and postoperative CT scans, co-registered with FreeSurfer and localized using the Versatile Electrode Localization Framework. Patients listened to 32 unique words presented via over-ear headphones (12 Hz-23.5 kHz bandwidth, 20 dB noise isolation). Each word lasted 700 ms with a 1000 ms inter-stimulus interval. A total of 120 stimuli were presented in a pseudorandom order. Neural signals were visually inspected to reject trials exhibiting artifactual activity. Signals were re-referenced using a common average reference spatial filter and segmented into individual trials from 1000 ms before to 1000 ms after auditory stimulus onset.

Two parameters were tested under HFES: stimulation frequency (10, 20, 50, 80, 100, 130 Hz) and interpolation duration (2, 4, 6, 8, 10, 20, 30 ms), constrained such that interpolation duration did not exceed 80% of the interval between consecutive stimulation pulses. Linear interpolation was applied to individual stimulation pulses during the auditory stimulus period (100-600 ms post-stimulus onset) and to the baseline period (600-100 ms pre-stimulus onset). Next, the denoised signals were filtered into the BHA range (70-170 Hz) using forward-backward filtering (pop_eegfiltnew in EEGLAB44). Then, the BHA was normalized by dividing each trial's amplitude by the standard deviation of the pre-stimulus period (−1000 to 0 ms) across all trials for each channel. The BHA envelope was extracted by computing the absolute value of the Hilbert transform of the normalized signal. For each trial, the mean BHA envelope was computed separately for the baseline and stimulation periods. BHA responses were compared between these periods using two sample t-tests, with false discovery rate (FDR) correction applied. Channels with significant differences (p<0.01) were labeled as auditory-responsive. The same analysis was performed on ground truth signals to extract the BHA, allowing direct validation by comparing LIBRA-denoised BHA to the original BHA. LIBRA validation was further extended to theta (4-7 Hz), alpha (8-12 Hz), low beta (13-20 Hz), high beta (20-30 Hz), and low gamma (30-50 Hz) frequency bands, following the same processing pipeline.

LIBRA was further validated under single pulse electrical stimulation (SPES), assuming a single stimulation pulse per trial at 300 ms after auditory stimulus onset (FIG. 4A). Linear interpolation was applied at the stimulation pulse and at a corresponding baseline period (300 ms pre-stimulus onset) using the same sets of interpolation duration as in HFES described above. Following artifact removal, the normalized BHA envelope was extracted from the denoised signals using the same procedure described above. Next, the baseline-corrected BHA trace was computed by subtracting the trial-averaged BHA trace in the baseline period (600 ms to 0 ms preceding stimulus onset) from the trial-averaged BHA trace in the stimulation period (0 ms to 600 ms following stimulus onset). Next focus was on LIBRA's temporal performance shortly after stimulation. Specifically, the post-interpolation period (0-25 ms) of the baseline-corrected BHA trace was divided into five 5 ms bins for each channel and compared the bin-wise BHA activity between the ground truth and denoised signals across channels.

In the synthetic analysis, focus was on evaluating the effectiveness of baseline correction. To isolate this aspect, it is assumed that the stimulation artifacts were restricted to the interpolation window, excluding additional artifact spread beyond this range. This assumption allowed us to systematically test whether baseline integration could mitigate interpolation-induced artifacts under controlled conditions. Additionally, while auditory stimulation-induced evoked responses were present in the ground truth signals, trial-averaged signal subtraction was not performed. This decision was made to intentionally retain signal fluctuations and better assess the robustness of the LIBRA method. A control analysis with trial-averaged subtraction yielded similar results, confirming that the presence of evoked responses did not confound LIBRA's artifact removal performance.

LIBRA was also compared with three existing methodologies for artifact removal (FIG. 6A, FIG. 6B). The first method (standard linear interpolation, SLI) substitutes the artifact-affected signal with a straight-line fit between the surrounding signals. The second method (physiological-informed interpolation, PII) reconstructs artifact-affected segments by merging reversed and tapered signal segments from both sides of the artifact window, preserving local physiological dynamics. The third method (baseline-integrated physiological interpolation, BIPI) extended the PII approach by applying it to both the stimulation and baseline periods, similar to LIBRA's baseline integration approach. These comparison methods represent commonly used strategies with varying levels of complexity and processing principles: SLI is a simple, widely used method; PII incorporates surrounding signal dynamics to improve data continuity; and BIPI integrates baseline data to provide a more direct benchmark for LIBRA.

To further evaluate LIBRA in clinical settings, it was applied to neural signals recorded from the frontal cortex during HFES and SPES of the basal and lateral amygdala. Given the anatomical and functional connectivity between the amygdala and frontal lobes, robust BHA responses were expected in frontal regions.

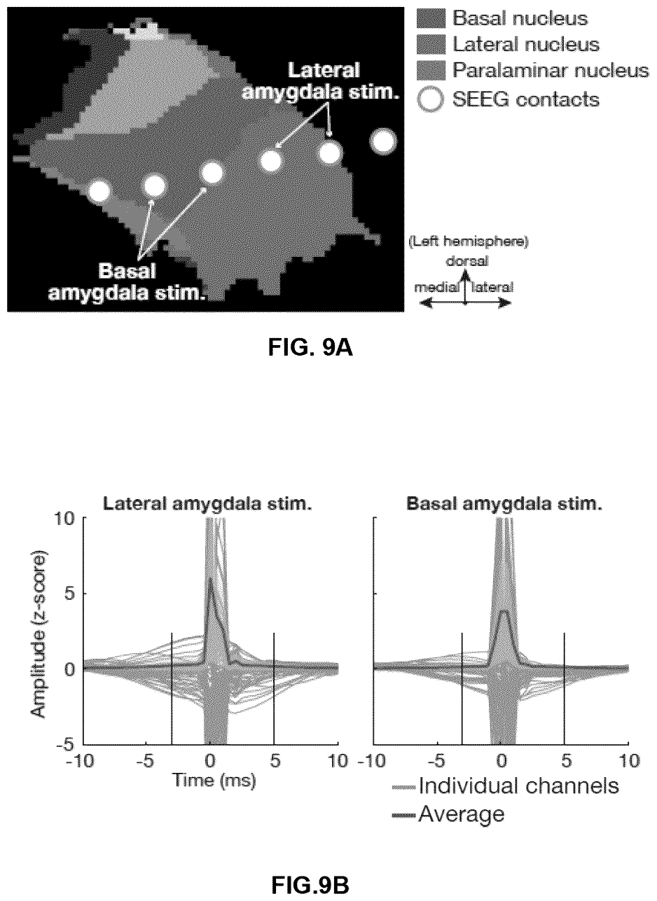

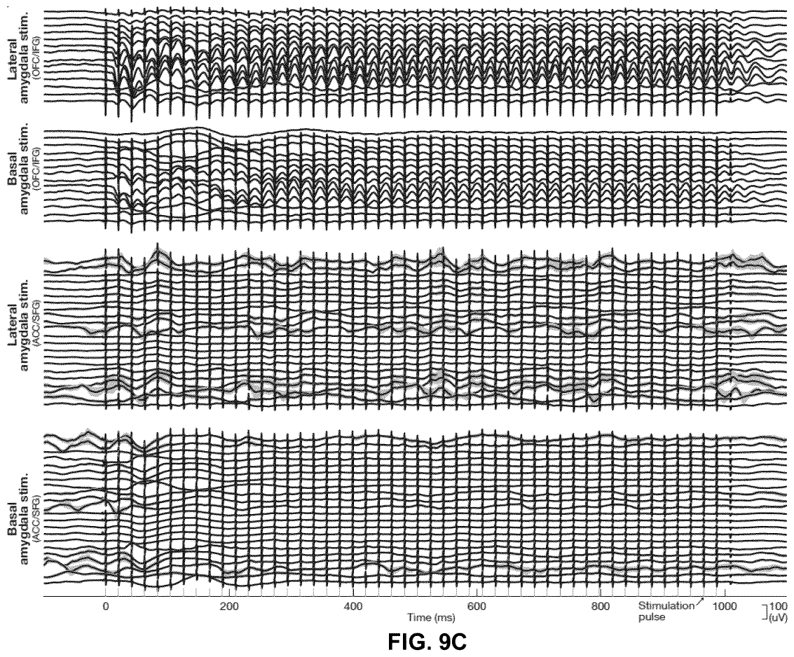

The signals were recorded from one human subject (male, aged 36) who underwent placement of SEEG electrodes for intractable epilepsy treatment. The study was approved by the institutional review board at Washington University in St. Louis, and informed consent was obtained from the subject. The recording settings were consistent with those described in the receptive language paradigm above. Stimulation was delivered to pairs of adjacent SEEG contacts within the basal and lateral amygdala (FIG. 9A). HFES (biphasic, pulse width 200 μs) was performed in 4 blocks for each stimulation pair, with 10 trials per block. Each trial consisted of 1000 ms of 50 Hz continuous stimulation followed by a 3000-5000 ms intertrial interval. The stimulation intensities for blocks 1-4 were 0.5, 1, 2, and 3 mA, respectively. SPES (0.5 Hz, biphasic, pulse width 200 μs, current amplitude 3 mA) was performed in 1 block of 60 s stimulation (30 trials). SEEG channels exhibiting artifactual activity due to broken contacts (e.g., no physiological signals) were excluded following visual inspection. The remaining signals were re-referenced using an average of signals from channels located at least 10 mm away from the stimulation sites. Individual trials were extracted from 1500 ms before to 1500 ms after stimulation onset for further analysis.

For HFES analysis, the trial-averaged signals were subtracted for each block and linear interpolation was applied to individual artifact pulses during the stimulation period (0 ms to 1000 ms after stimulation onset) and the baseline period (1100 ms to 100 ms before stimulation onset). Based on the observed characteristics of the stimulation artifacts (FIG. 9B), each linear interpolation was designed to span 3 ms before and 5 ms after each pulse onset. The BHA envelope was extracted using the same method as in the synthetic analysis. Mean BHA activity was calculated for the stimulation and baseline periods, and a randomization test was employed to robustly assess the significance of BHA responses. Specifically, mean BHA values for each channel were correlated with condition labels (baseline=−1, stimulation=1) using Spearman's rank correlation coefficient (r). The resulting r values were tested against a null distribution generated by 1000 random permutations of the condition labels, with significance determined through FDR correction. Additionally, the mean BHA was correlated during the stimulation period with stimulation intensities, applying the same randomization and FDR correction process to determine significance. Channels showing significant correlations (p<0.01) with both the condition labels and stimulation intensity were defined as responsive.

For SPES analysis, the LIBRA methodology was utilized by subtracting trial-averaged signals for each block and applying linear interpolation (3 ms before and 5 ms after pulse onset) to artifact pulses occurring at the stimulation onset (0 ms). Baseline interpolation was performed over the 8 ms window at 400 ms before stimulation onset. The BHA envelope was computed using the Hilbert transform, and the baseline-corrected BHA trace was calculated by subtracting the trial-averaged BHA trace in the baseline period (500-100 ms before stimulation onset) from the stimulation period (100 ms before to 300 ms after stimulation onset).

### Results

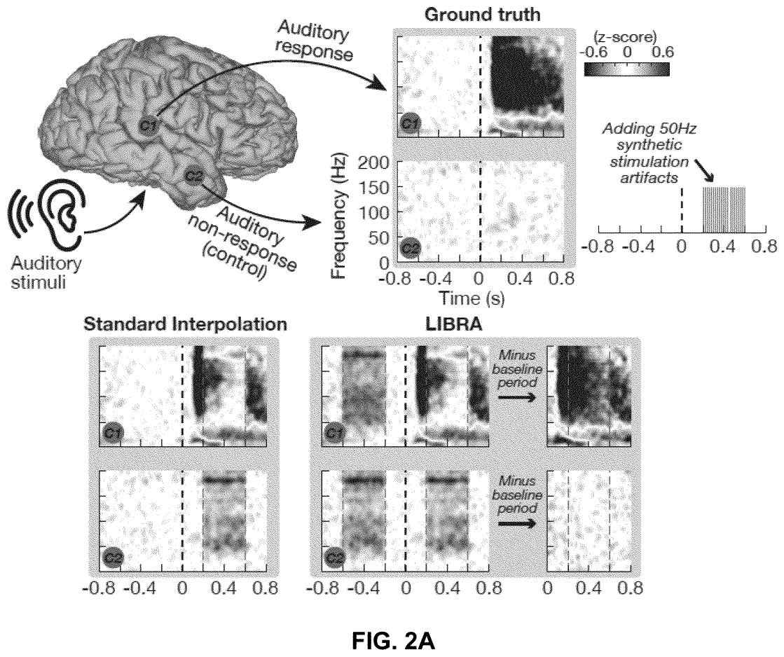

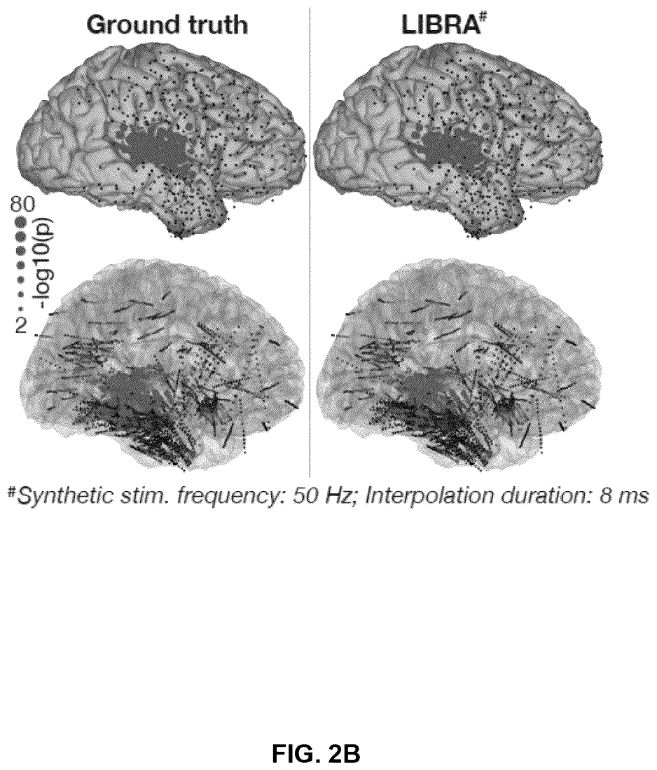

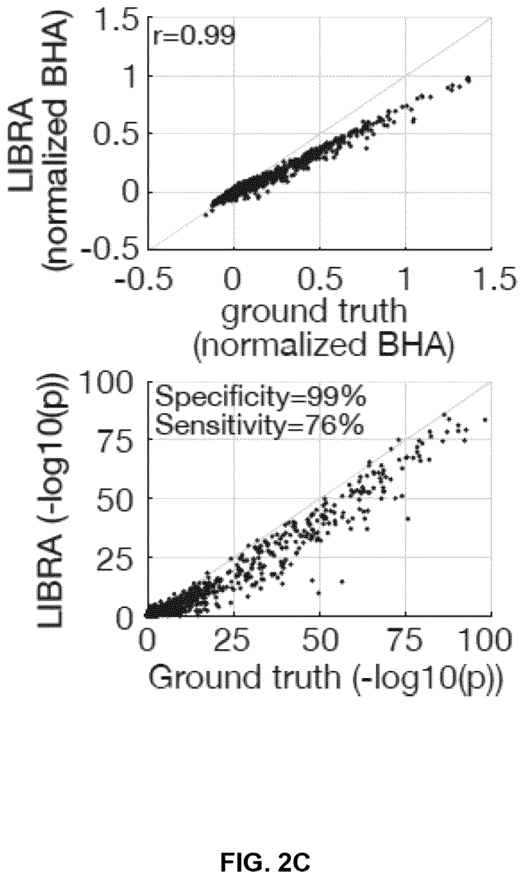

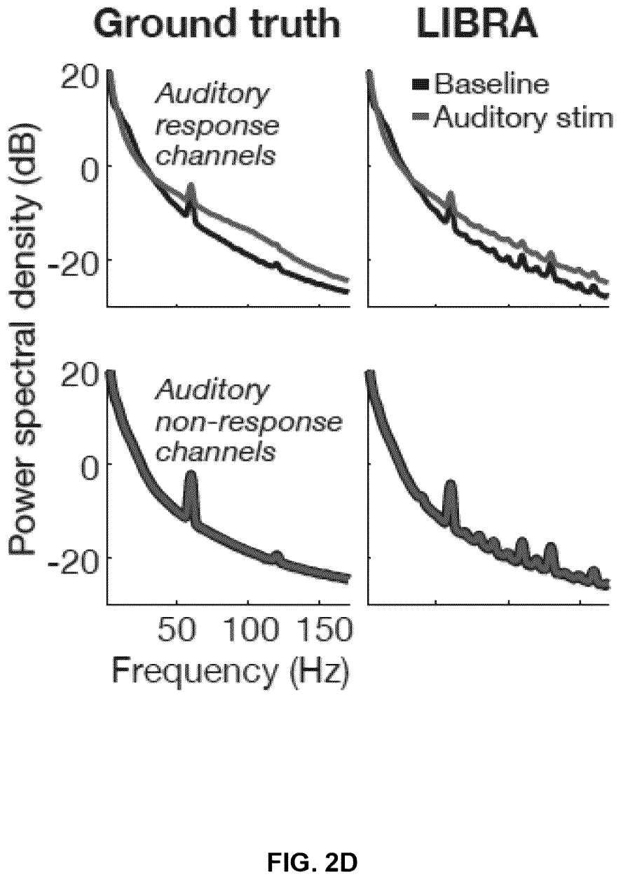

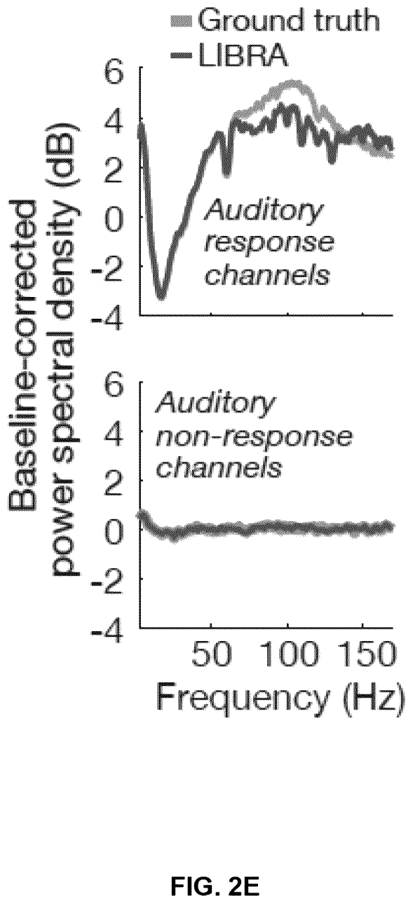

To intuitively evaluate LIBRA's performance, studies began with a representative example using synthetic 50 Hz stimulation and an 8 ms interpolation duration, a common and challenging scenario due to overlap with the BHA range. As shown in FIG. 2A, time-frequency plots from two representative SEEG channels revealed that standard interpolation retained broadband spectral artifacts that mimicked BHA. In contrast, LIBRA effectively eliminated these artifacts following baseline subtraction. The example channels were located in Heschl's gyrus and middle temporal cortex, demonstrating LIBRA's robustness across auditory-responsive and non-responsive sites.

Topographical analysis further revealed that LIBRA effectively preserved auditory-related BHA responses. The denoised signals closely matched the ground truth signals, as evidenced by the high overlap in the spatial distribution of

BHA (FIG. 2B) and strong correlation in both BHA response amplitude (FIG. 2C; top) and BHA significance (FIG. 2C; bottom). The spectral analysis provided further insights into LIBR's efficacy: while linear interpolation introduced observable interpolation-related artifacts in the power spectral density during both the stimulation and baseline periods (FIG. 2D), the baseline-corrected power was remarkably similar between the denoised and ground truth signals (FIG. 2E). This demonstrates that by leveraging baseline subtraction, LIBRA effectively mitigated interpolation-related artifacts, isolating the true neurophysiological responses.

### Validation of LIBRA During High-frequency Electrical Stimulation (HFES)

To systematically evaluate LIBRA's robustness in isolating BHA, seven interpolation durations (2-30 ms) were tested across six synthetic stimulation frequencies (10-130 Hz) and compared its performance with three commonly used artifact removal methods. Under a representative condition of 50 Hz stimulation with an 8 ms interpolation duration, LIBRA exhibited the highest correlation between denoised and ground truth BHA responses (FIG. 3A). Sensitivity analysis confirmed LIBRA's superior ability to correctly detect responsive channels, while specificity analysis demonstrated its reliability in rejecting non-responsive channels (FIG. 3B).

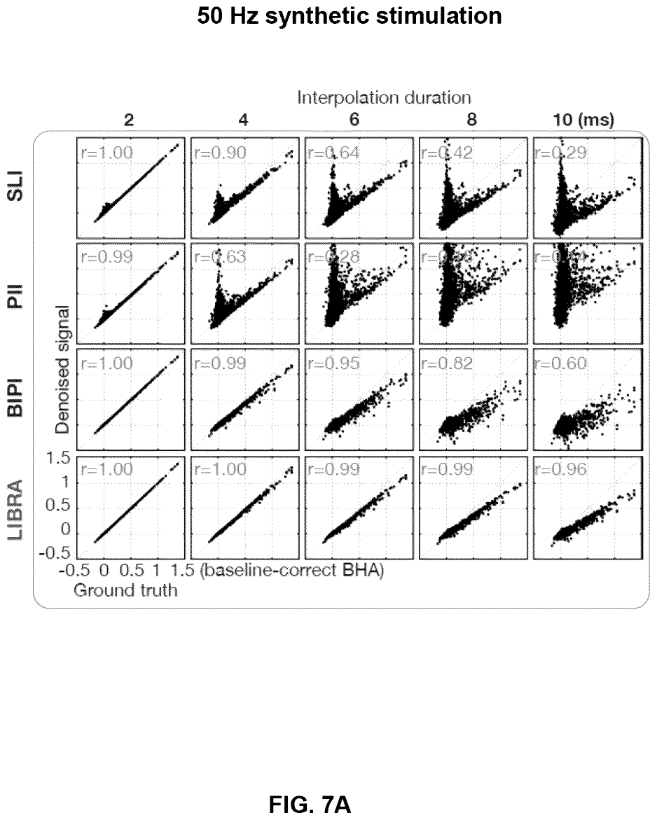

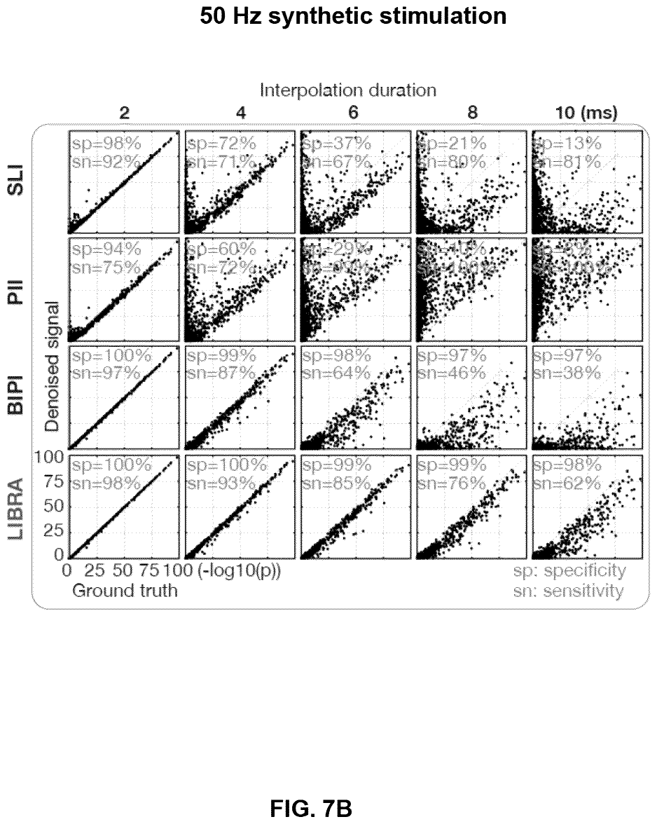

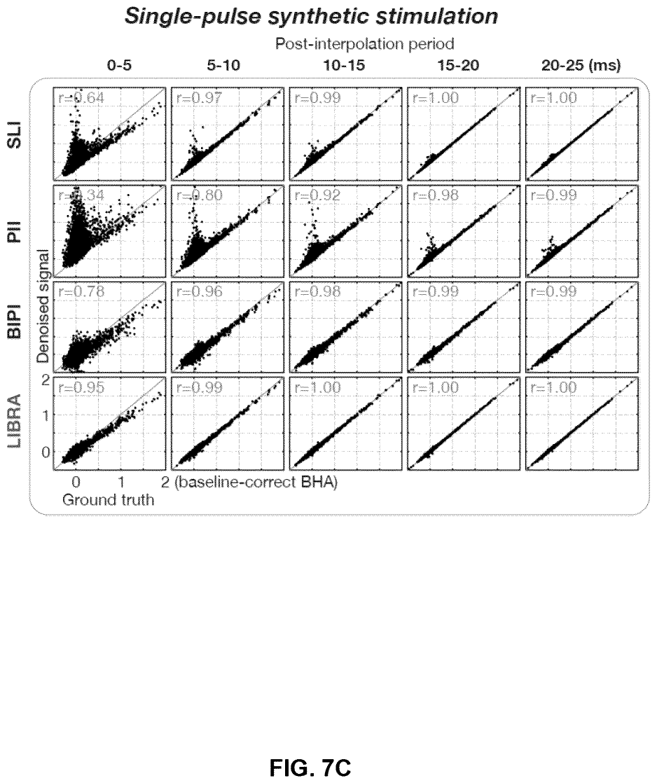

More broadly, LIBRA consistently outperformed the alternative methods across all tested conditions (FIG. 3C, FIG. 7A, FIG. 7B, FIG. 7C). Summary metrics averaged across stimulation frequencies and interpolation durations further demonstrated LIBRA's robust and consistent performance, with significantly higher correlation values compared to BIPI (p=3.9×10⁻³), PII (p=1.2×10⁻⁸), and SLI (p=1.3×10⁻⁶) (FIG. 3D).

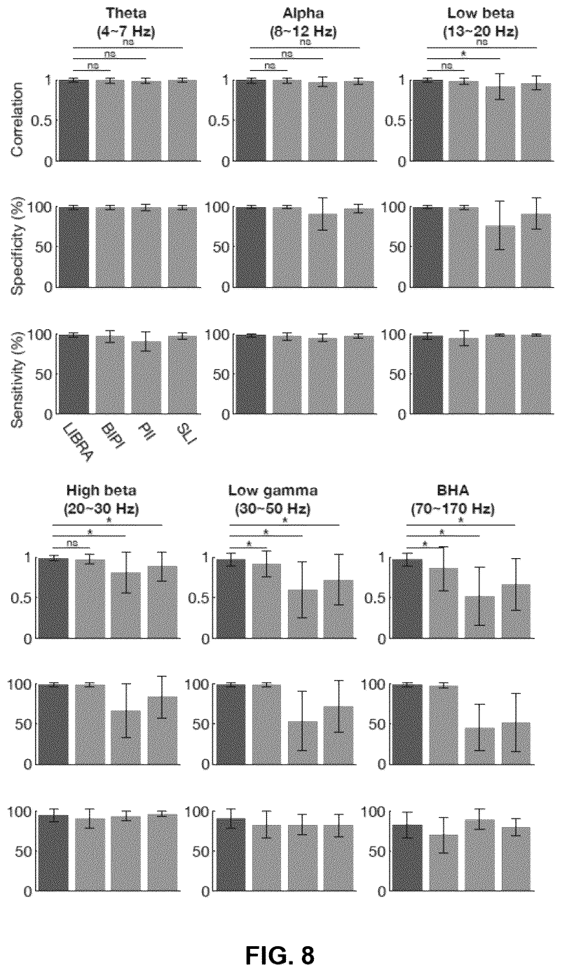

LIBRA's performance was further evaluated across canonical frequency bands to ensure its efficacy in preserving neurophysiological activity beyond the BHA range (FIG. 8). LIBRA demonstrated comparable performance to alternative methods in lower-frequency bands, including theta (4-7 Hz) and alpha (8-12 Hz). However, in higher frequency bands, such as low gamma (30-50 Hz) and BHA (70-170 Hz), LIBRA consistently outperformed the other methods. These results underscore LIBRA's ability to effectively remove stimulation artifacts while preserving accurate neurophysiological responses across a wide range of canonical frequencies.

### Validation of LIBRA During Single-Pulse Electrical Stimulation (SPES)

To intuitively evaluate LIBRA's performance under SPES, representative stimulation parameters were applied as a single stimulation pulse per trial with an 8 ms interpolation duration. After artifact removal with LIBRA, BHA activity was computed for auditory-related response and non-response channels. The resulting BHA trace confirmed that LIBRA effectively eliminated stimulation artifacts while preserving distinct temporal patterns of BHA response (FIG. 4A).

To evaluate LIBRA's performance immediately following artifact removal, the correlation between ground truth and denoised BHA signals was calculated in the earliest post-interpolation bin (0-5 ms). LIBRA achieved a high correlation of r=0.95, indicating strong preservation of neural responses immediately after interpolation. By contrast, alternative methods showed substantially lower correlations: SLI (r=0.64), PII (r=0.34), and BIPI (r=0.78) (FIG. 4B).

To comprehensively evaluate LIBRA's performance, all interpolation durations (2-30 ms) were tested across each post-interpolation time window (FIG. 4C, FIG. 4D). LIBRA consistently yielded the highest correlations between denoised and ground truth BHA responses, significantly outperforming BIPI (p=1.7×10⁻³), PII (p=9.5×10⁻⁵), and SLI (p=1.5×10⁻³). These results validate LIBRA's effectiveness in preserving fine-grained neurophysiological dynamics during artifact removal in SPES.

### Validation of LIBRA for Amygdala-induced Prefrontal Neural Modulation

LIBRA's ability to extract neurophysiological responses was validated during HFES (50 Hz) and SPES (0.5 Hz) by analyzing BHA recorded from the frontal lobe in response to basal and lateral amygdala stimulation (FIG. 9B).

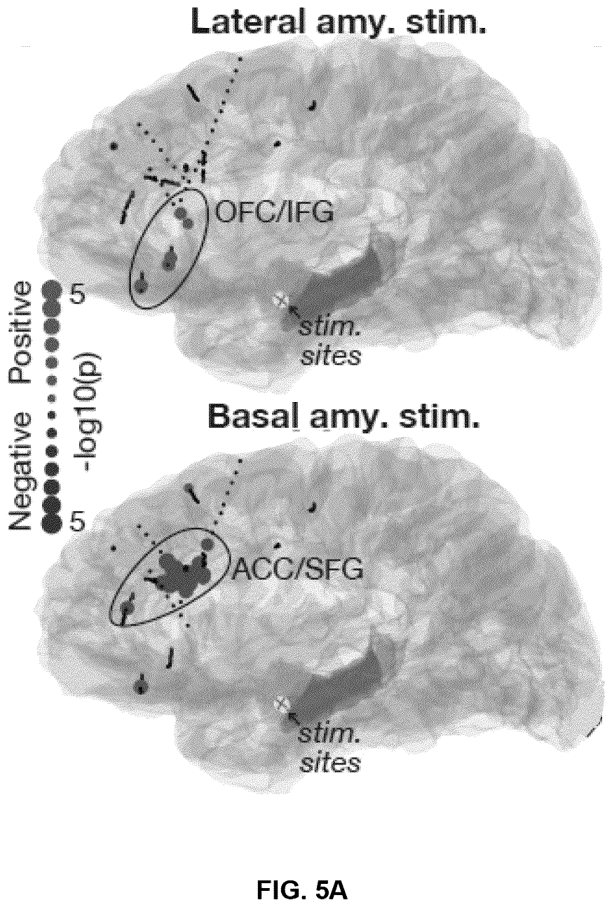

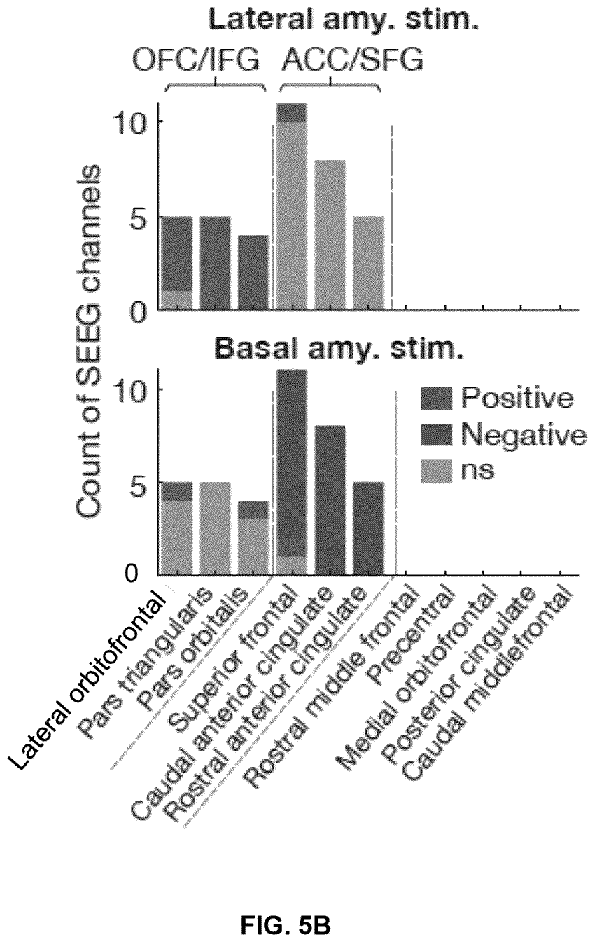

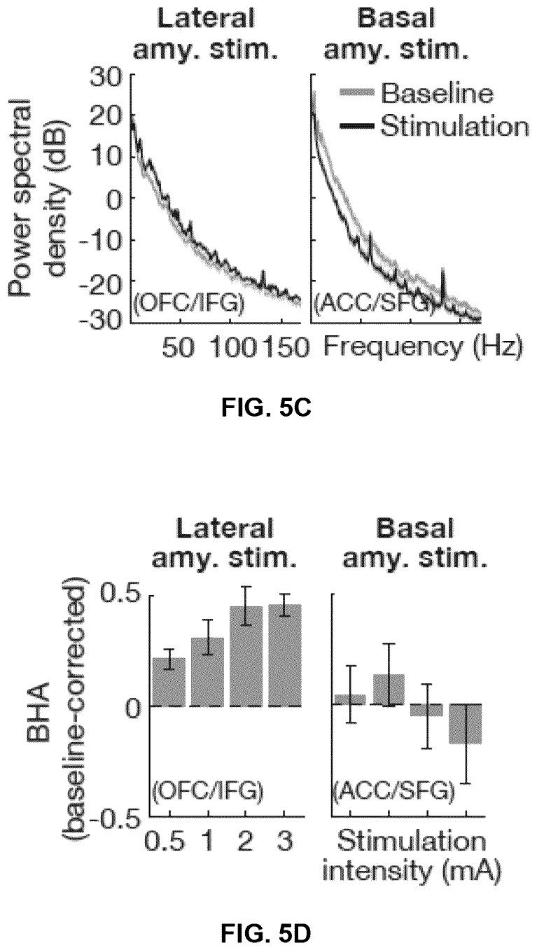

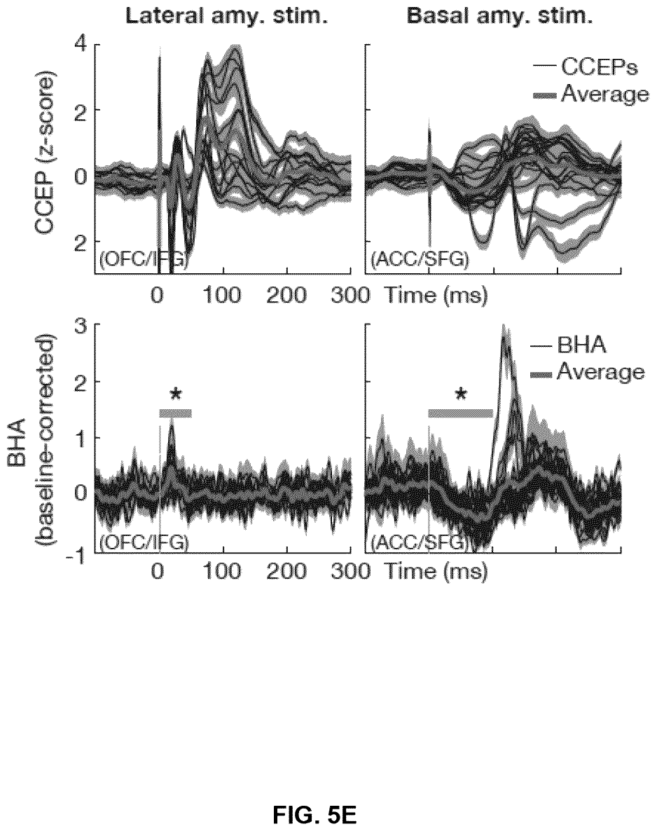

During 50 Hz stimulation, lateral amygdala stimulation induced positive correlations between BHA and stimulation intensities in the orbitofrontal cortex (OFC) and inferior frontal gyrus (IFG), while basal amygdala stimulation resulted in negative correlations in the anterior cingulate cortex (ACC) and superior frontal gyrus (SFG) (FIG. 5A, FIG. 5B). Power spectral density analyses revealed clear spectral differences between the baseline and stimulation periods, reflecting distinct neural responses to lateral versus basal amygdala stimulation (FIG. 5C). Furthermore, lateral amygdala stimulation led to a dose-dependent increase in BHA power in the OFC/IFG, while basal amygdala stimulation produced a dose-dependent decrease in BHA power in the ACC/SFG (FIG. 5D). These findings are consistent with human resting-state neuroimaging studies, which report a positive correlation between the amygdala and ventromedial prefrontal cortex and a negative correlation with the dorsal anterior cingulate cortex.

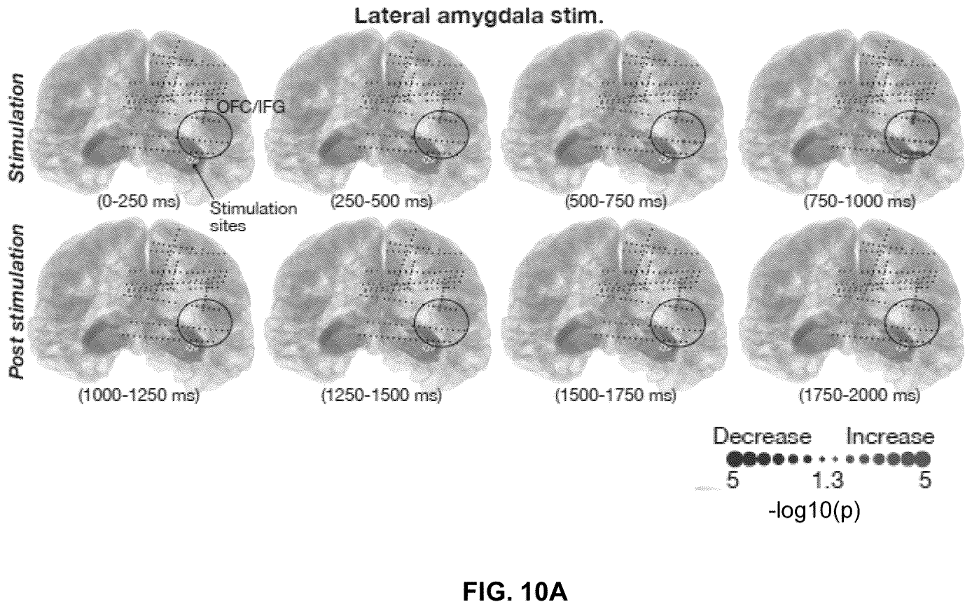

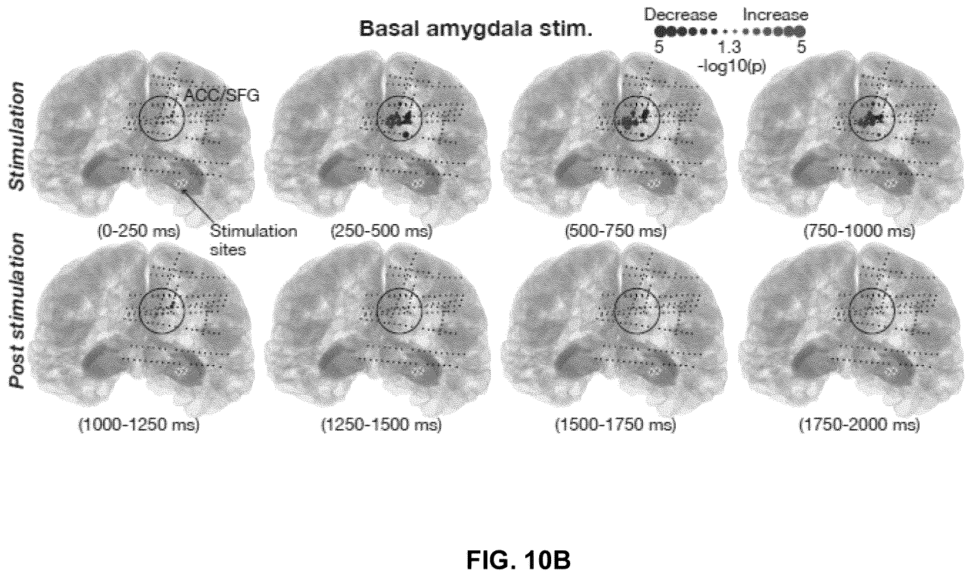

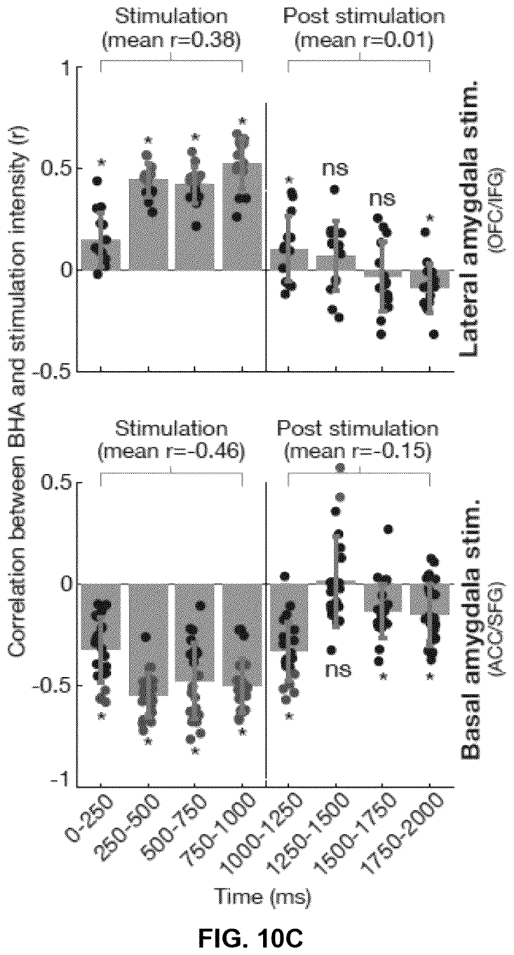

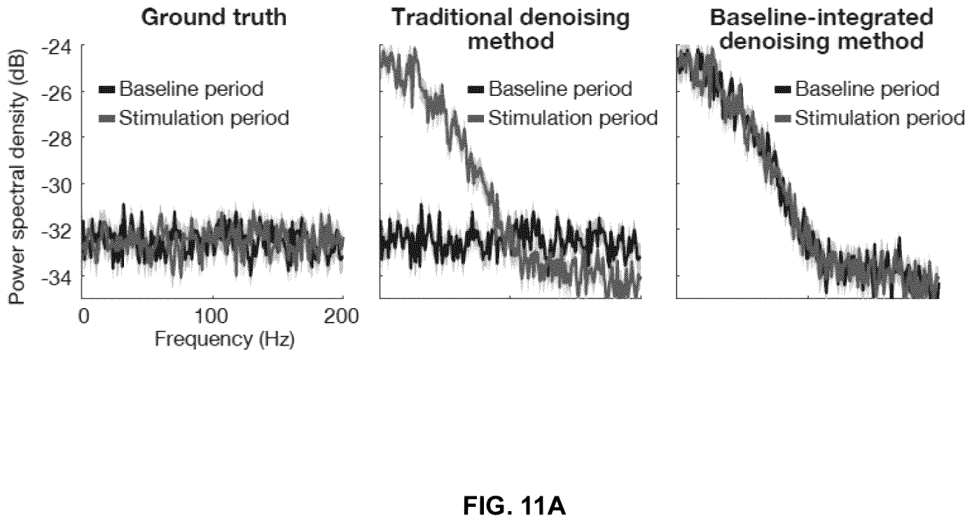

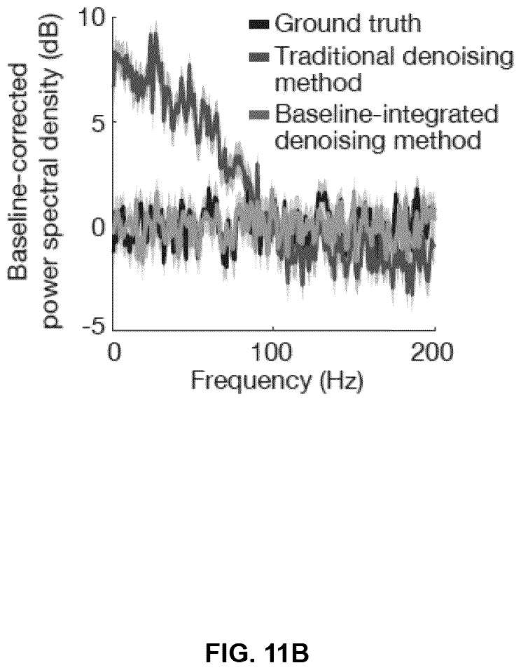

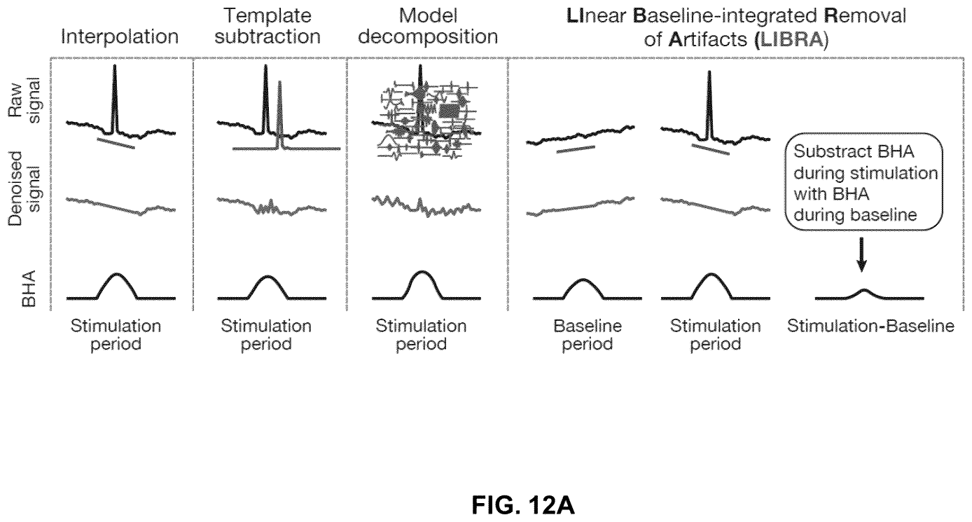

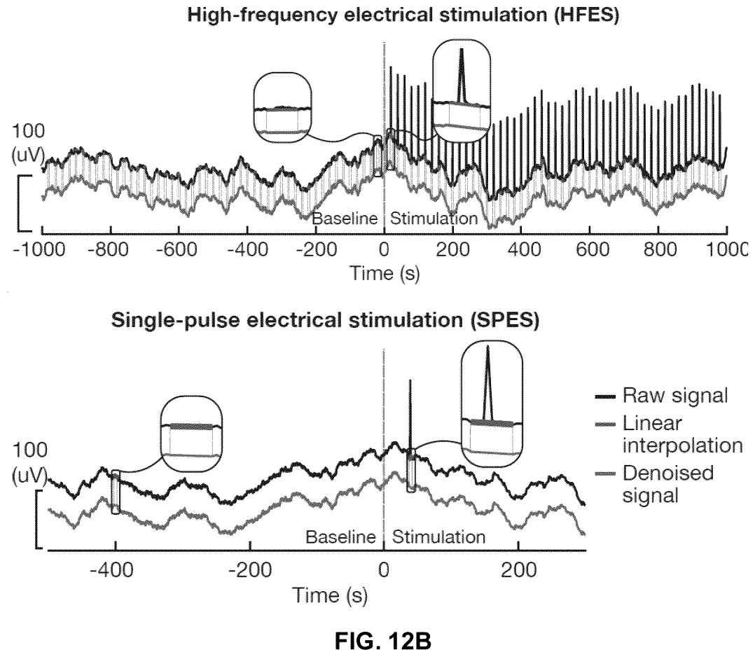

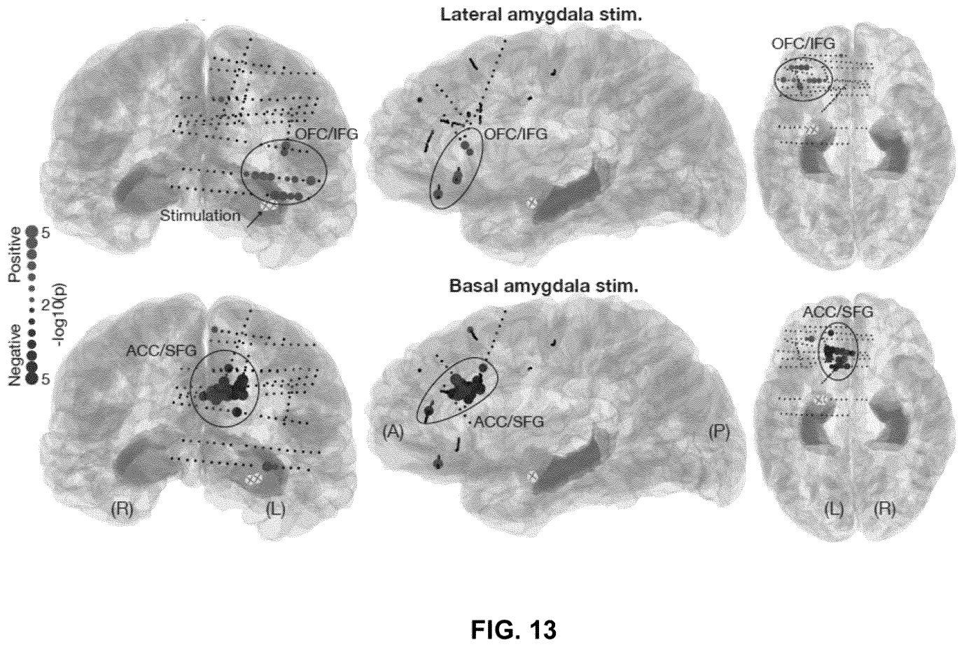

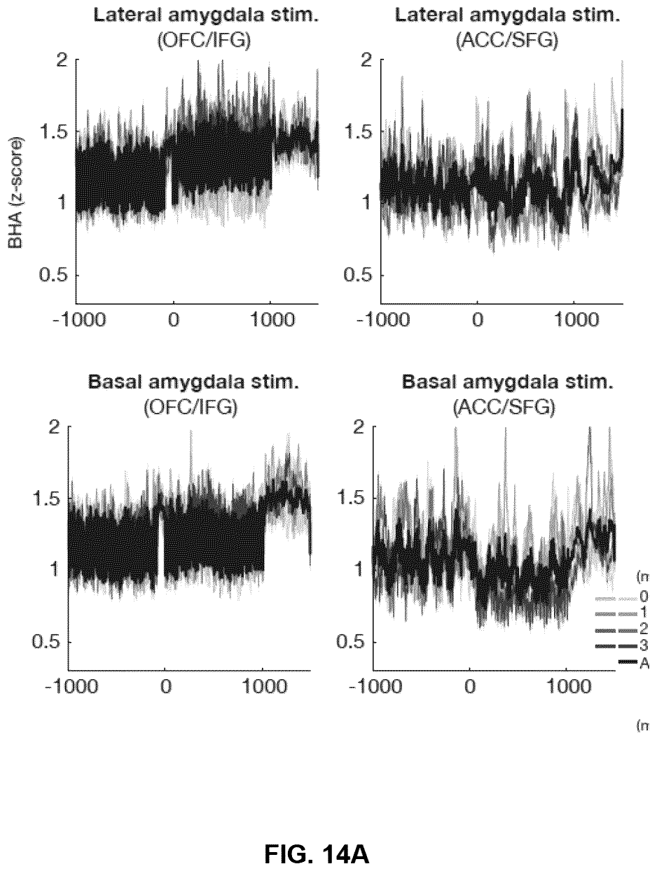

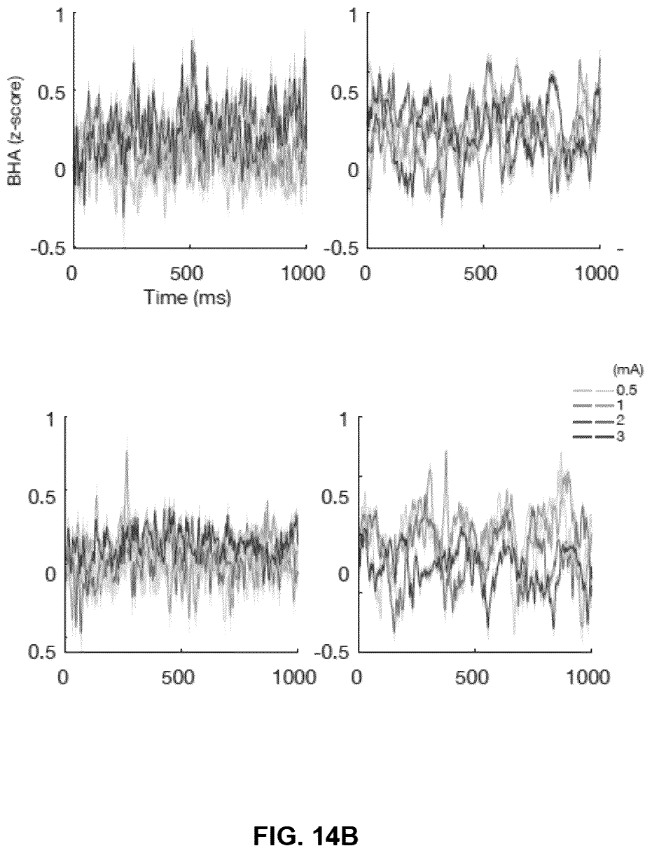

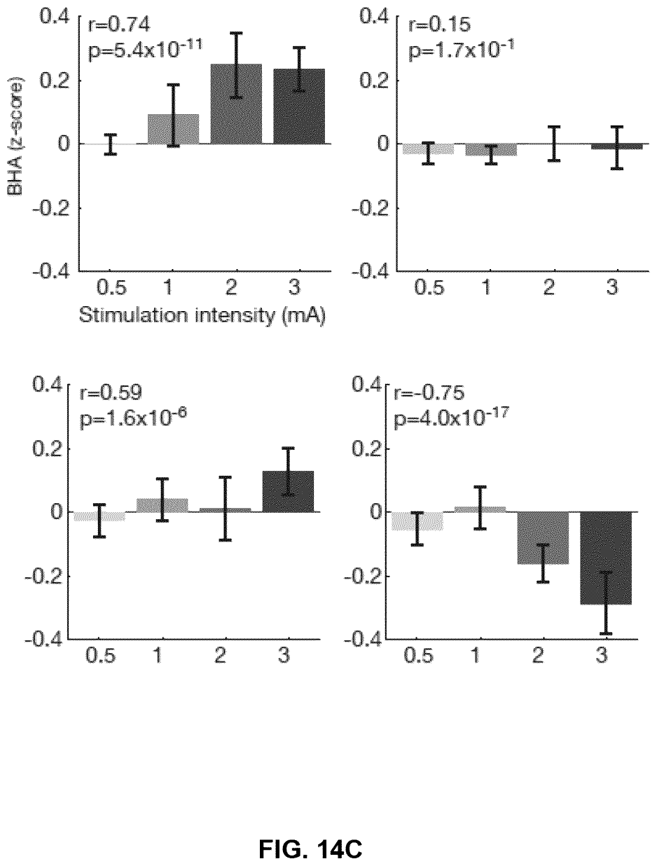

To evaluate the temporal dynamics of BHA responses, baseline-corrected BHA was analyzed in 250 ms time bins spanning the stimulation (0 -1000 ms) and post-stimulation (1000-2000 ms) periods (FIG. 10A, FIG. 10B, FIG. 10C). The analysis revealed only moderate modulation of BHA by the stimulation intensity during the initial stimulation period (0-250 ms), while stronger modulation was observed during the later stimulation period (e.g., after 250 ms). Notably, significant modulation at the group level persisted into the post-stimulation period, although the modulation strength was markedly reduced. These results highlight the temporal specificity of neural responses to amygdala stimulation and underscore LIBRA's capability to capture ongoing neural dynamics during high-frequency electrical stimulation.

Further analyzed was cortico-cortical evoked potentials (CCEPs) and baseline-corrected BHA induced by SPES in regions showing significant modulation during 50 Hz stimulation. Distinct CCEP morphologies were observed in OFC/IFG and ACC/SFG under lateral and basal amygdala stimulation, respectively, highlighting region-specific neural responses (FIG. 5E; top). More importantly, significant BHA modulation occurred shortly after SPES (FIG. 5E; bottom). Specifically, lateral amygdala stimulation increased BHA in OFC/IFG within 50 ms post-stimulation (p=2.3×10⁻⁴, one-sample two-tailed t-test), while basal amygdala stimulation decreased BHA in ACC/SFG within 100 ms poststimulation (p=1.5×10⁻⁷, one-sample two-tailed t-test). These results align with the dose-dependent responses observed during 50 Hz stimulation, confirming the consistency of neural response patterns across stimulation frequencies.

### Discussion

LIBRA provides a robust framework for extracting neurophysiological responses during electrical stimulation by effectively removing artifacts while preserving BHA. By integrating baseline correction with linear interpolation, LIBRA mitigates interpolation-induced distortions and reliably isolates true neural activity, even when stimulation frequencies overlap with the BHA range.

LIBRA is designed to extract non-phase-locked responses, such as the BHA that reflects population-level neural dynamics. The linear interpolation during the LIBRA denoising process does not fundamentally alter the overall structure of the non-phase-locked signal because it primarily replaces short, artifact-affected segments while leaving the majority of the signal untouched. Non-phase-locked neural responses are characterized by their inherent variability and broad spectral content, which are not strictly time-locked to the stimulation. Therefore, as long as the interpolation spans only a brief duration relative to the entire signal, it minimally disrupts the global structure and dynamics of the non-phase-locked responses. This aligns with the findings that LIBRA denoising performance decreases as the length of the interpolation increases, a trend observed across all artifact removal approaches (FIG. 7A). Moreover, the use of linear interpolation ensures a smooth transition across the replaced segment, maintaining continuity in the signal. This minimizes the risk of introducing abrupt changes or artifacts that could distort the broader spectral or temporal characteristics of the non-phase-locked response. This approach maintains the overall signal structure, enabling LIBRA to reliably extract meaningful neurophysiological responses.

The focus on un-interpolated signal periods explains LIBRA's relatively higher specificity compared to sensitivity (FIG. 3A, FIG. 3B, FIG. 3C, FIG. 3D). High specificity arises because LIBRA's artifact removal process is conservative, prioritizing the exclusion of spurious signals over the inclusion of ambiguous responses. This means LIBRA is highly effective in correctly identifying non-responsive channels, reducing the likelihood of false positives. On the other hand, the relatively lower sensitivity reflects a trade-off inherent in LIBRA's design. Some subtle or weak neural responses that might overlap temporally with artifact-affected periods could be partially suppressed during the interpolation process. While this conservatism may lead to missed detections (lower sensitivity), it ensures the reliability of the detected signals, which is crucial for high-stakes applications like intracranial recordings.

The findings from the amygdala stimulation study reveal that neural responses during stimulation are significantly stronger and more reliable than those observed post-stimulation (FIG. 10C). By reliably extracting neural responses during stimulation, LIBRA unlocks a novel avenue for investigating ongoing neural dynamics during high-frequency electrical stimulation. This capability enables researchers to probe the immediate excitatory or inhibitory effects of stimulation on specific brain regions (FIG. 5A), uncovering direct causal pathways that were previously obscured by artifacts. Additionally, the ability to analyze neural activity during stimulation provides a powerful tool for studying dynamic interactions within neural circuits, such as modulation of connectivity or plasticity, and can inform the optimization of neuromodulation therapies.

What is claimed is:

1. A computer-implemented method to remove stimulation artifacts from a series of signal sets indicative of electrophysiological activity obtained during at least one stimulation trial, the method comprising:
a. subtracting, using a computing device, a trial-averaged signal from each individual stimulation trial of the at least one stimulation trial;
b. interpolating, using the computing device, the series of signals within a baseline period and interpolating within a stimulation period containing the stimulation artifacts for each individual stimulation trial of the at least one stimulation trial;
c. extracting, using the computing device, a non-phased-locked response from the series of signals for each individual stimulation trial of the at least one stimulation trial;
d. subtracting, using the computing device, a baseline spectral response within the baseline period from a stimulation spectral response within the stimulation period to produce a neurophysiological signal set for each individual stimulation trial of the at least one stimulation trial; and
e. displaying, using the computing device, the neurophysiological signal set for each individual stimulation trial of the at least one stimulation trial, wherein each neurophysiological signal set corresponds to each signal set of the series of signal sets with stimulation artifacts removed.

2. The method of claim 1, wherein the method of interpolation of the baseline period and the method of interpolation of the stimulation period are independently selected from a linear interpolation method, a Crowther method, a polynomial curve fitting method, a matching pursuit method, and a wavelet interpolation method.

3. The method of claim 1, wherein the electrical brain stimulation is performed at a stimulation frequency comprising theta (4-7 Hz), alpha (8-12 Hz), low beta (13-20 Hz), high beta (20-30 Hz), low gamma (30-Hz), broadband high-frequency activity (BHA; 70-170 Hz), and any combination thereof.

4. A computer-implemented method of monitoring an efficacy of a neuromodulation therapy by transforming a series of signal sets indicative of electrophysiological activity obtained during at least one stimulation trial associated with the neuromodulation therapy into a neurophysiological signal set for each individual stimulation trial of the at least one stimulation trial, the method comprising:
a. subtracting, using a computing device, a trial-averaged signal from each individual stimulation trial of the at least one stimulation trial;
b. interpolating, using the computing device, the series of signals within a baseline period and interpolating within a stimulation period containing the stimulation artifacts for each individual stimulation trial of the at least one stimulation trial;
c. extracting, using the computing device, a non-phased-locked response from the series of signals for each individual stimulation trial of the at least one stimulation trial;
d. subtracting, using the computing device, a baseline spectral response within the baseline period from a stimulation spectral response within the stimulation period to produce a neurophysiological signal set for each individual stimulation trial of the at least one stimulation trial; and
e. displaying, using the computing device, the neurophysiological signal set for each individual stimulation trial of the at least one stimulation trial, wherein each neurophysiological signal set corresponds to each signal set of the series of signal sets with stimulation artifacts removed;
wherein the each neurophysiological signal set is indicative of the efficacy of the neuromodulation therapy.

5. The method of claim 4, wherein the method of interpolation of the baseline period and the method of interpolation of the stimulation period are independently selected from a linear interpolation method, a Crowther method, a polynomial curve fitting method, a matching pursuit method, and a wavelet interpolation method.

6. The method of claim 4, wherein the method preserves a temporal signal, a spectral signal, and a region-specific neural response within the measured neurophysiological signals during the electrical stimulation.

7. The method of claim 4, wherein the electrical stimulation is a constant-current square-wave delivered at an interval from 0.1 ms to 1 ms and a frequency from 0.5 Hz to 250 Hz.
